(19) United States
(12) Patent Application Publication
(10) Pub. No.: US 2025/0304915 A1
(43) Pub. Date: Oct. 2, 2025

(54) Myeloid Cells Overexpressing BCL2

(71) Applicants: Institut Curie, Paris (FR); INSERM (Institut National de la Santé et de la Recherche Médicale), Paris (FR)

(72) Inventors: Nicolas Manel, Paris (FR); Xavier Lahaye, Cachan (FR); Jaime Fuentealba, Gentilly (FR); Michael Saitakis, Paris (FR); Sebastian Amigorena, Paris (FR)

(73) Assignees: Institut Curie, Paris (FR); INSERM (Institut National de la Santé et de la Recherche Medicalé), Paris (FR)

(21) Appl. No.: 18/563,599

(22) PCT Filed: May 25, 2022
(86) PCT No.: PCT/EP2022/064297

§ 371 Date: Nov. 22, 2023

(30) Foreign Application Priority Data
May 25, 2021 (EP) 21305680.7

(51) Int. Cl. C12N 5/0786; A61K 40/17; A61K 40/31; A61K 40/42; C07K 14/705; C07K 16/28

(52) U.S. Cl. CPC C12N 5/0645; A61K 40/17; A61K 40/31; A61K 40/4202; A61K 40/4211; C07K 14/705; C07K 16/2803; C12N 2510/00

(57) ABSTRACT

The present invention relates to isolated modified myeloid cell, a progenitor thereof, or a progeny thereof, encoding an antigen recognizing receptor, wherein said myeloid cell or progenitor thereof has been further modified to overexpress BCL2. The present invention further relates to therapeutic application in particular in the field of adoptive therapy of said modified myeloid cells.

### FIELD OF THE INVENTION

The present invention relates to the field of adoptive therapy. The present invention provides immune myeloid cells overexpressing BCL2 with enhanced survival potential and expressing antigen recognizing receptor.

### INTRODUCTION

Adoptive T cell therapy (ATCT) using T cells armed with recombinant T Cell Receptor (TCR) and Chimeric Antigen Receptor (CAR) technologies is emerging as a powerful cancer therapy alternative (Lim W A & June C H. 2018. Cell 168 (4): 724-740).

In particular, CAR T immunotherapy has demonstrated profound results in haematological malignancies, while clinical efficacy in the solid tumours remain poorly observed. Barriers to T cell entry and function may partially explain this difference. Optimization of CAR therapy can be addressed either via CAR design or through the choice of the cellular vessels expressing the CAR.

The solid tumor environment however actively recruits myeloid cells. In particular, macrophages are abundant in TME where they often facilitate metastasis and promote tumour progression by increasing immunosuppression. Thus therapeutic approaches to deplete or repolarize tumor associated macrophages (TAMs), which express activating and inhibiting Fc receptors and are polarized toward a pro-tumoral and immunosuppressive M2 phenotype are currently envisioned. However, myeloid cells, and macrophages in particular are critical effectors of the innate immune system with effector function such as phagocytosis, cellular toxicity and secretion of pro-inflammatory molecules and antigen presentation to T cells, such that they can have an important role in promoting adaptive anti-tumor responses. Also, dendritic cells are antigen presenting cells that serves as functional link between innate and adaptive immunity, being thus crucial for antigen-specific T cell responses. Indeed given that a suitable antigen is available, DC modified to express this antigen can drive T cells against viral or cancer cells and initiate further immune response.

Thus, myeloid cells, including dendritic cells and macrophages are promising cells for use in adoptive cell therapy of cancer. They are however characterized by their short life in cell culture and in vivo. The reasons for death of these cells overtime remains unknown. There is therefore an important clinical need to develop tools that extend the survival of myeloid cells and their progeny in vivo in order to increase their therapeutic effect, in particular their anti-tumor effect.

### SUMMARY

The inventors have unexpectedly discovered that myeloid cells transduced with the anti-apoptotic factor Bcl2, optionally expressing a chimeric antigen receptor, exhibit a long-term in vivo survival (or persistence) after adoptive transfer. The observed survival effect was increased and more reliable as compared to the use of known anti-apoptotic drugs. It was found that while BCL2 overexpression increases survival but not cellular integrity in dendritic cells, it unexpectedly increases cellular integrity but not survival in macrophages, in particular macrophages expressing a chimeric antigen receptor. Such modified myeloid cells are therefore of high relevance in the field of adoptive therapy. Also, the modified cells typically expressing an antigen recognizing receptor such as a CAR, display enhanced efficacy for treating cancer.

Therefore, the present invention relates to an isolated modified myeloid cell, a progenitor thereof, or a progeny thereof, encoding an antigen recognizing receptor, wherein said myeloid cell or progenitor thereof has been further modified to overexpress BCL2.

The present invention also includes the use of an isolated modified myeloid cell, a progenitor thereof, or a progeny thereof for use in a vaccination strategy.

Typically, the progenitor is a myeloid progenitor, a granulocyte monocyte dendritic cell progenitor (GMDP), a monocyte dendritic cell progenitor (MDP) or a common dendritic cell progenitor (cDP), notably a granulocyte, a monocyte, a macrophage or a dendritic cell having targeted effector activity.

Typically, the targeted effector activity is directed against an antigen on a target cell that is specifically bound by the antigen recognizing receptor. The targeted effector activity can be selected from the group consisting of phagocytosis, targeted cellular cytotoxicity, antigen presentation, cytokine secretion, and activation of cell migration.

The antigen recognizing receptor is generally recombinantly expressed in the modified myeloid cells as herein described, optionally it is a chimeric antigen receptor.

The antigen recognizing receptor can be expressed from a vector, optionally a viral vector, in particular a lentivirus vector, optionally an HIV-1-derived viral vector.

In some embodiments, the expression of SIRPa in the modified myeloid cell as herein described is disrupted or downregulated.

Typically, the antigen recognized by the antigen receptor, typically a CAR, is a tumor antigen, which can be for example selected from the group consisting of CD19, MUC16, MUC1, CA1X, CEA, CD8, CD7, CD 10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD3, ITER-2, hTERT, IL-13R-a2, K-light chain, KDR, LeY, LI cell adhesion molecule, MAGE-A1, Mesothelin, ERBB2, MAGEA3, p53, MARTI,GPI00, Proteinase3 (PR1), Tyrosinase, Survivin, hTERT, EphA2, NKG2D ligands, NY-ES0-1, oncofetal antigen (h5T4), PSCA, PSMA, ROR1, TAG-72, VEGF-R2, WT-I, BCMA, CD123, CD44V6, NKCS1, EGF1R, EGFR-VIII, and CD99, CD70, ADGRE2, CCR1, LILRB2, PRAME, CCR4, CD5, CD3, TRBC1, TRBC2, TIM-3, Integrin B7, ICAM-I, CD70, Tim3, CLEC12A and ER.

In embodiments, wherein the antigen recognizing receptor is a CAR, said receptor can comprise a signalling domain selected from CD3, the y subunit of Fc receptor (such as of FcaRly), CD64, CD32, CD32b, CD32c, CD16, CD16a, CD16b, MEGF10, CD40, the Toll-like receptor/Interleukin (IL)-1 receptor (TLR/IL-1R) superfamily, members of the BAI family of phosphatidylserine receptor, such as BAI1, and members from the TAM family of phosphatidylserine receptors, such as MerTK; optionally wherein the CAR comprises a TLR signalling domains including the Toll/interleukin receptor homology domain and any intracellular domains interacting with the MyDDosome and/or theTRIFosome clusters such as in particular with MyD88, TIRAP, TRIF and/or TRAM). The present invention also relates to a pharmaceutical composition comprising at least a modified myeloid cell or a progenitor thereof as herein disclosed and a pharmacological excipient.

The present invention also pertains to a method for producing a modified myeloid cell or a progenitor thereof, the method comprising a step consisting in expressing or increasing the expressing of bcl2 in a myeloid cell or a progenitor thereof; and further comprising a step consisting in recombinantly expressing in said cell an antigen recognizing receptor.

The present invention also encompasses the therapeutic application of a modified cell myeloid cell or a progenitor as herein described or a composition comprising thereof. In particular the present invention encompasses the use of a modified cell myeloid cell or a progenitor as herein described or a composition comprising thereof in the treatment of cancers, auto-immune diseases, or infectious diseases.

The modified cell myeloid cell or a progenitor as herein described or a composition comprising thereof are of particular relevance for adoptive cell therapy in a subject in need thereof. Typically, in such embodiments, the myeloid cell or progenitor thereof is autologous.

### BRIEF DESCRIPTION OF THE FIGURES

FIG. 1. A: BCL2 overexpression inhibit MVA-induced apoptosis in MDCCs. Electron micrographs of non-infected MDDCs (NI, scale bar 10 μm), non-transduced (NT, scale bar 2 μm), BCL2 expressing MDDCs (BLC2, scale bar 5 μm). Representative images are derived from 3 donors obtained in two independent experiments. In all graphs 0.3 and 1 indicate MOI of MVA. NI=non-infected. B-C: FSC/SSC characteristics, LIVE/DEAD labeling, and CD86/Siglec-1 expression of MDDCs transduced with a control vector (black bar) or with a BCL2 encoding vector (grey bar) 24 hpi with MVA. Statistical analysis on the right. n=8 donors from four independent experiments.

FIG. 2. BCL2 overexpression provides a better survival of myeloid cells in vivo. Graphs show the average radiance (p/see/cm2/Sr) measured on days 2, 5 and 12 after adoptive transfer (n=3 mice per condition), both from ventral (top) and dorsal (bottom) views.

FIG. 3. BCL2 overexpression provides a better survival of myeloid cells in vivo expressing different CARs. Graphs show the ventral average radiance (p/see/cm2/Sr) measured on days 1, 5, 9 and 13 after adoptive transfer (n=3 mice per condition). The different CAR constructs expressed by the infused myeloid cells are: CD19trunc (top graph), CD19z (middle graph) and CD19BBz (bottom graph).

FIG. 4. BCL2 overexpression enhances the integrity of MDMs and CAR-MDMs. A: Total cell numbers and cell integrity were measured on days 4, 7, 10, 17, 24 and 32 after monocyte transduction and MDDCs differentiation in control MDDCs (BFP-2A (•)) or BCL2 expressing MDDCs (BFP-2A-BCL2 (▪)) (n=2 donors, paired repeated measures (RM) one-way ANOVA with a Sidak's post-test, line at mean). B: Cell numbers and cell integrity were measured on days 10, 17, 24, and 32, after monocyte transduction and MDMs differentiation in control MDMs (BFP-2A (•)) or BCL2 expressing MDMs (BFP-2A-BCL2 (▪)) (n=4 donors (only n=3 at D34); unpaired repeated measures (RM) one-way ANOVA with a Sidak's post-test, line at mean). C: Cell numbers and cell integrity were measured on days 10, 17, 24 after monocyte transduction and MDMs differentiation in control MDMs (BFP-2A+ pCDH1-CAR-CD3 (•)) or BCL2 expressing MDMs (BFP-2A-BCL2+ pCDH1-CAR-CD3 (▪)) (n=2 donors, paired repeated measures (RM) one-way ANOVA with a Sidak's post-test, line at mean).

### DETAILED DESCRIPTION

The presently disclosed subject matter provides modified myeloid cells and progenitors thereof expressing or overexpressing bc/2 and expressing a receptor recognizing a target antigen. The presently disclosed subject matter also provides methods of obtaining such cells, as well as methods of using them for inducing and/or enhancing an immune response to the target antigen, and/or treating and/or preventing a cancer or tumor or other diseases/disorders where an increase in an antigen-specific immune response is desired. The presently disclosed subject matter is based, at least in part, on the discovery that myeloid cells modified to express or to overexpressed bcl2 have increased in vivo survival (i.e. in vivo persistence) or increased cellular integrity (in case of macrophages) as compared to myeloid cells that do not overexpress bcl2 (typically which to not express bcl2), and exhibit improved therapeutic potency (e.g., increased tumor infiltration) compared to control cells which are not expressing bcl2.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et ak, Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold of a value.

The terms “comprises”, “comprising”, and are intended to have the broad meaning and can mean “includes”, “including” and the like.

By “activates an immunoresponsive myeloid cell” it is meant an induction of a signal transduction or changes in protein expression in the cell resulting in initiation of an immune response. For example, activation of a myeloid cell may involve activation of an intracellular cascade inducing detectable cell proliferation and/or leading to the initiation of effector functions. Activation of immunoresponsive myeloid cells can thus be associated with induced cytokine production, phagocytosis, cell signalling, target cell killing, or antigen processing and presentation.

Typically, in response to ligand biding to an antigen recognizing receptor, a signal transduction cascade is produced. In certain embodiments, when a recombinantly expressed CAR binds to an antigen, a transduction cascade is activated such that an immune response is initiated.

The term “antigen-recognizing receptor” as used herein refers to a receptor that is capable of activating a myeloid immunoresponsive cell in response to its binding to an antigen. Non-limiting examples of antigen-recognizing receptors include chimeric antigen receptors (“CARs”).

The term “chimeric antigen receptor” or “CAR” as used herein refers to a molecule comprising an extracellular antigen-binding domain that is fused to an intracellular signalling domain that is capable of activating or stimulating a myeloid cell as herein defined, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signalling domain. In certain embodiments, the CAR is selected to have high binding affinity or avidity for the antigen.

The term “antibody” used herein should be intended in the broadest sense and includes polyclonal and monoclonal antibodies, including full antibodies and functional (antigen-binding) antibody fragments, including fragment antigen binding (Fab) fragments, F(ab′) 2 fragments, Fab′ fragments, Fv fragments, recombinant lgG (rlgG) fragments, variable heavy chain (VH) regions capable of specifically binding the antigen, single chain antibody fragments, including single chain variable fragments (scFv), and single domain antibodies (e.g., sdAb, sdFv, nanobody) fragments. Unless otherwise stated, the term “antibody” should thus be understood to encompass functional antibody fragments thereof. The term also encompasses intact or full-length antibodies, including antibodies of any class or sub-class, including IgG and sub-classes thereof, IgG1, IgG2, IgG3, IgG4, IgM, IgE, IgA, and IgD and any origin (such as human camelid or other). In some embodiments the antibody comprises a heavy chain variable region and a light chain variable region. The term “antibody” encompasses whole native antibodies but also recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In certain embodiments, an antibody is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as VL) and a light chain constant CL region. The light chain constant region is comprised of one domain, CL. The VH and VL regions can be further sub-divided into regions of hypervariability, termed complementarity determining regions (CDR), interspersed with regions that are more conserved, termed framework regions (FR). Each VH and VL IS composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system.

As used herein, “CDRs” are defined as the complementarity determining region amino acid sequences of an antibody which are the hypervariable regions of immunoglobulin heavy and light chains. See, e.g., Rabat et ak, Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Rabat system (Rabat, E. A., et al. (1991) Sequences of Proteins of Immunological Interest, Fifth Edition, ET.S. Department of Health and Human Services, NIH Publication No. 91-3242).

An “antibody fragment” refers herein to a molecule other than a full antibody that comprises a portion of a full antibody that binds the antigen to which the full antibody binds. Examples of antibody fragments include but are not limited to Fv, Fab, Fab′, Fab′-SH, F(ab′)2; diabodies; linear antibodies; variable heavy chain (VH) regions, VHH antibodies, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

As used herein, the term “single-chain variable fragment” or “scFv” is a fusion protein of the variable regions of the heavy (VH) and light chains (VL) of an immunoglobulin covalently linked to form a VH:: VL heterodimer. The VH and VL are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See also U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27 (6): 455-51; Peter et al., J Cachexia Sarcopenia Muscle 2012 Aug. 12; Shieh et al., J Imunol2009 183 (4): 2277-85; Giomarelli et al., Thromb Haemost 2007 97 (6): 955-63; Fife eta., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chem 2003 25278 (38): 36740-7; Xie et al., Nat Biotech 1997 15 (8): 768-71; Ledbetter et al., Crit Rev Immunoll997 17 (5-6): 427-55; Ho et al., BioChim Biophys Acta 2003 1638 (3): 257-66).

“Single-domain antibodies” as used herein are antibody fragments comprising all or a portion of the heavy chain variable domain or all or a portion of the light chain variable domain of an antibody. In certain embodiments, a single-domain antibody is a human single-domain antibody.

The term “antigen” or “Ag” as used herein meant a molecule that provokes an immune response. This immune response may involve either antibody production, or the activation of specific immunoresponsive cells, or both. It must be understood that any macromolecule, including virtually all proteins or peptides, can serve as an antigen. Furthermore, antigens can be derived from recombinant or genomic DNA. Thus, any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. The term “tumor antigen” as used herein refers to any polypeptide expressed by a tumor that is capable of inducing an immune response.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

By “specifically binds” is meant a polypeptide or fragment thereof that recognizes and binds a polypeptide of interest, but which does not substantially recognize and bind other molecules in a sample, for example, a biological sample, which naturally includes a polypeptide of the invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” it is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

The term “constitutive expression” or “constitutively expressed” as used herein refers to expression or expressed under all physiological conditions.

As used herein, the term “promoter/regulatory sequence” means a nucleic acid sequence recognized by the synthetic machinery of the cell, or introduced synthetic machinery which is required for expression of a gene product operably linked to the promoter/regulatory sequence. In some instances, this sequence may be the core promoter sequence and in other instances, this sequence may also include an enhancer sequence and other regulatory elements which are required for expression of the gene product. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By “increased” it is meant positively altered by at least about 5%. An alteration may be by about 5%, about 10%, about 25%, about 30%, about 50%, about 75%, about 100% or more.

By “reduced” is meant negatively altered by at least about 5%. An alteration may be by about 5%, about 10%, about 25%, about 30%, about 50%, about 75%, or even by about 100%.

By “reference” or “control” is meant a standard of comparison. For example, in the present invention, the level of bcl2 expression in a myeloid cell modified to express or overexpress bcl2 can be compared to the level of bcl2 expression in the corresponding non-modified myeloid cell.

By “isolated cell” is meant a cell that is separated from the molecular and/or cellular components that naturally accompany the cell. The terms “isolated,” “purified,” or “biologically pure” used herein generally refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “disease” is meant any condition or disorder that damages or interferes with the normal function of a cell, tissue, or organ. Examples of diseases include neoplasia or pathogen infection of cell.

The terms “tumor” or “neoplasia” are used interchangeably and mean a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia or tumor can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, melanomas, leukemias, or plasmacytomas (malignant tumor of the plasma cells). Illustrative neoplasms or cancer for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors or cancers such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendo-theliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

By “effective amount” is meant an amount sufficient to arrest, ameliorate, or inhibit the continued proliferation, growth, or metastasis (e.g., invasion, or migration) of a neoplasia.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, the term “autologous” is meant to refer to any material derived from the same individual to which it is later to be re-introduced into the individual.

Other aspects of the presently disclosed subject matter are described in the following disclosure and are within the ambit of the presently disclosed subject matter.

### 2. Immunoresponsive Myeloid Cells

### 2.1 Type of Cells

The cells according to the invention are typically mammalian cells, e.g., human cells.

An immunoresponsive cell as herein intended refers to a cell, a progenitor, or progeny thereof that is part of the immune system and helps the body to fight infections and other diseases such as cancer. More particularly, the cells of the invention are myeloid cells of the innate immune system (i.e., immune cells) that are differentiated descendants from common progenitors derived from hematopoietic stem cells in the bone marrow. Myeloid cells typically include granulocytes (more particularly neutrophils), monocytes, macrophages, and dendritic cells (DCs) and their progenitors. More particular phenotypes of these myeloid cells and their progenitors according to the present invention are described below.

In some embodiments, the myeloid cells include one or more subsets of a myeloid cell population such as whole immunoresponsive cell population in peripheral blood, for example the whole dendritic cell population including their conventional DCs (cDCs) and plasmacytoid subset cell populations and subpopulations thereof, such as those defined by function, activation state, maturity, potential for differentiation, expansion, recirculation, localization, and/or persistence capacities, antigen-specificity, type of antigen-specific receptor, presence in a particular organ or compartment, marker or cytokine secretion profile, and/or degree of differentiation. The sub-types and subpopulations of myeloid cells according to the present invention are described herein after in details.

Typically, the myeloid cell according to the present invention possesses targeted effector activity. More particularly, the myeloid cell has targeted effector activity directed against an antigen on a target cell, such as through specific binding to an antigen binding domain of a CAR. In some embodiments, the targeted effector activity includes, but is not limited to phagocytosis, targeted cellular cytotoxicity, antigen presentation, cytokine secretion, and/or activation of cell migration.

Phagocytosis may be assessed using a flow cytometric method for example utilizing a CellTracker dye which typically allows to measure the phagocytosis efficiency of cancer cells by phagocytic cells such as macrophages (Nam, Gi-Hoon et al. “An optimized protocol to determine the engulfment of cancer cells by phagocytes using flow cytometry and fluorescence microscopy.” Journal of immunological methods vol. 470 (2019): 27-32).

Targeted cellular cytotoxicity assays are for example described in Rabinovich, Peter M et al. “A versatile flow-based assay for immunocyte-mediated cytotoxicity.” Journal of immunological methods vol. 474 (2019): 112668. Such assays may involve loading target cells with any reported compound such as compounds that change fluorescence after killing, like time-resolved fluorescence resonance energy transfer (TR-FRET) probes and calcein. Another set of assays, which measures the release of constitutively expressed molecules like lactose dehydrogenase, adenylate kinase, and glyceraldehyde 3-phosphate dehydrogenase can also be used. Measurement of luciferase released from target cells modified to genetically express the luciferase is further highly sensitive. Indirect methods such as gamma interferon secretion by T cells also fast and high throughput analysis of effector cell activation. In still another method target cells can be fluorescently labelled with a non-toxic, cell-permeable dye that covalently binds to cell proteins, including nuclear proteins. The labelled target cells are then incubated with effector cells to begin killing. Following the killing reaction, the cell mixture can be incubated with another dye that specifically stains proteins of dead cells, including nuclear proteins. In the final step, cell nuclei are released (for example using Triton X-100) and analyzed by flow cytometry.

Antigen presentation may be assessed by using anti-HLA class II antibodies. Cells may be identified by flow cytometry.

Secretion of cytokine can be assessed using specific antibodies directed against the selected cytokine. Numerous commercial kits of secretion assays are now at the disposal of the skilled person.

Activation of cell migration or motility, notably dendritic cell migration can be typically assessed by determination of CC-chemokine receptor 7 (CCR7) expression, notably with an increase of CCR7 expression as compared to non-activated, immature DCs. Indeed, upregulation of CCR7 is associated with dendritic cell activation and increase of motility. The CCR7 ligand CC-chemokine ligand 21 (CCL21) is expressed on terminal lymphatics and CCR7-CCL21 interactions enable DCs to enter the lymphatic vasculature and eventually the draining lymph node, where they migrate into the T cell-rich paracortex (Worbs, T., Hammerschmidt, S. & Förster, R. Dendritic cell migration in health and disease. Nat Rev Immunol 17, 30-48 (2017).

In some embodiments, the cells include one or more subsets of myeloid cells or other cell types, such as whole dendritic cell and subpopulations thereof, such as those defined by function, activation state, maturity, potential for differentiation, expansion, recirculation, localization, and/or persistence capacities, antigen-specificity, type of antigen-specific receptor, presence in a particular organ or compartment, marker or cytokine secretion profile, and/or degree of differentiation.

### 2.1.1 Myeloid Progenitors

Hematopoietic stem cells (HSCs) develop through various multipotent progenitor stages into monocyte/macrophage and dendritic progenitors (MDP). Hematopoietic stem and progenitor cells can be obtained, for example, from cord blood, or from peripheral blood, e.g. peripheral blood-derived CD34+ cells after mobilization treatment with granulocyte-colony stimulating factor (G-CSF). Typically, a CD34+ hematopoietic precursor gives rise to myeloid (MP) and lymphoid (LP) precursors such as the common myeloid progenitor (CMP). CMP have been characterized as Lin⁻, CD34⁺, CD38⁺, IL3Ra^{low} and CD45A. CMP differentiate into monocytes, macrophages and DC precursors (MDP), which will give rise to monocytes and to the common DC precursors (CDP). MDP have lost the ability to generate granulocytes and either give rise to “common monocyte progenitors” (cMoPs) restricted to monocytes and their descendants or commit toward a common DC precursor (cDP). MDPs have been characterized as lineage negative (LIN-), CD135+ (FLT3), CD117⁺ (KIT) and CD115⁺ (CSF1R).

cDP are typically described as giving rise to classical DC (CDC) and plasmacytoid DC (pDC) via common DC precursors (CDPs) (Ginhoux F and Jung S 2014, Fogg D K et al. 2006). Their phenotype has been described as CD135⁺, CD115⁺ and DNGR-1⁺. Pre-cDC have been described as the progenitors of the two major cDC subpopulations named cDC1 and cDC2. Precursors that can be used for in vitro generation of DCs can be extracted from the bone marrow. In the presence of GM-CSF, these precursors give rise to large number of cells that resemble tissue DC and are called bone marrow-derived dendritic cells (BMDC).

cMoPs give rise to monocytes and their derivatives, but do not generate pDC or cDC. Typically, cMoPs phenotypically differ from MDPs in that they do not express CD135 (Hettinger J et al. 2013). They may be typically characterized by a CD115⁺, CD135⁻ and Ly6C⁺ phenotype.

The granulocyte/monocyte progenitor (GMP) gives rise to all granulocytes through further differentiation into the eosinophil lineage-committed progenitor (EoP), and the basophil/mast cell progenitor (BMCP), which in turn gives rise to the mast cell progenitor (MCP) and the basophil progenitor (BaP). It has been shown that GMP could be characterized by Lin⁻, CD34⁺, CD38⁺, IL3Ra⁺ and CD45A⁻ expression.

GM-CSF stimulates growth and differentiation of granulocyte and monocyte/macrophage precursor cells. It is also known to affect the function of mature myeloid cells by priming monocytes and neutrophils for enhanced adhesion, tumour cytotoxicity, or leukotriene production (see Gamble J R, Elliott M J, Jaipargas E, Lopez A F, Vadas M A. “Regulation of human monocyte adherence by granulocyte-macrophage colony-stimulating factor.” Proc Natl Acad Sci USA. 1989; 86:7169-73; Grabstein K H, Urdal D L, Tushinski R J, et al. «Induction of macrophage tumoricidal activity by granulocyte-macrophage colony-stimulating factor.” Science. 1986; 232:506-8; and Dahinden C A, Zingg J, Maly F E, de Week A L. “Leukotriene production in human neutrophils primed by recombinant human granulocyte/macrophage colony-stimulating factor and stimulated with the complement component C5A and FMLP as second signals.” J Exp Med. 1988; 167:1281-95). GM-CSF together with IL-4 promotes the differentiation of monocytes into dendritic cells in vitro (see Sallusto F, Lanzavecchia A. “Efficient presentation of soluble antigen by cultured human dendritic cells is maintained by granulocyte/macrophage colony-stimulating factor plus interleukin 4 and downregulated by tumor necrosis factor alpha.” J Exp Med. 1994; 179:1109-18; and Chapuis F, Rosenzwajg M, Yagello M, Ekman M, Biberfeld P, Gluckman J C. “Differentiation of human dendritic cells from monocytes in vitro.” Eur J Immunol. 1997; 27:431-41).

In some embodiments of the present application, the myeloid cell is a myeloid progenitor, typically a MDP or a progeny thereof as above defined. Typically, a myeloid cell progenitor can be selected from HSCs, MPs, CMPs, MDPs, CDPs, cMoPs, cDPs, or GMPs. In some embodiments, the progenitor is a multipotent myeloid progenitor, notably selected from MPs, CMPs, MDPs, CDPs, cMoPs, cDPs, or GMPs.

For reviews regarding myeloid cells and their progenitors see for example Weiskopf, Kipp et al. “Myeloid Cell Origins, Differentiation, and Clinical Implications.” Microbiology spectrum vol. 4,5 (2016): 10.1128/microbiolspec.MCHD-0031-2016. doi: 10.1128/microbiolspec.MCHD-0031-2016; Schraml B U, Reis e Sousa C. Defining dendritic cells. Curr Opin Immunol. 2015; 32:13-20. doi: 10.1016/j.coi.2014.11.001 or Viola A, Munari F, Sánchez-Rodríguez R, Scolaro T, Castegna A. The Metabolic Signature of Macrophage Responses. Front Immunol. 2019; 10:1462; Geissmann, Frederic et al. “Development of monocytes, macrophages, and dendritic cells.” Science (New York, N.Y.) vol. 327,5966 (2010): 656-61. doi: 10.1126/science.1178331; Weiskopf, Kipp et al. “Myeloid Cell Origins, Differentiation, and Clinical Implications.” Microbiology spectrum vol. 4,5 (2016): 10.1128/microbiolspec.MCHD-0031-2016. doi: 10.1128/microbiolspec.MCHD-0031-2016; Patente, Thiago A et al. “Human Dendritic Cells: Their Heterogeneity and Clinical Application Potential in Cancer Immunotherapy.” Frontiers in immunology vol. 9 3176. 21 Jan. 2019, doi: 10.3389/fimmu.2018.03176; Shapouri-Moghaddam, Abbas et al. “Macrophage plasticity, polarization, and function in health and disease.” Journal of cellular physiology vol. 233,9 (2018): 6425-6440. doi: 10.1002/jcp.2642.

### 2.1.2 Monocytes

In some embodiments of the present application, the myeloid cell as herein defined is a monocyte. Monocytes develop in the bone marrow and represent the primary type of mononuclear phagocyte found in the blood. Monocytes are members of the mononuclear phagocyte system (MPS), which is a comprehensive classification of all highly phagocytic mononuclear cells and their precursors (van Furth R, Cohn Z A. The origin and kinetics of mononuclear phagocytes. J Exp Med. 1968 Sep. 1;128 (3): 415-35., van Furth, R et al. “The mononuclear phagocyte system: a new classification of macrophages, monocytes, and their precursor cells.” Bulletin of the World Health Organization vol. 46,6 (1972): 845-52.). It comprises all myeloid immune cells other than polymorphonuclear granulocytes. Classical models of monocyte differentiation propose that circulating monocytes originate from hematopoietic stem cell (HSC)-derived progenitors with myeloid restricted potential (Geissmann F et al. Science 2010, see above). Subsequent commitment steps during monocyte differentiation in the bone marrow involve common myeloid progenitors (CMPs), granulocyte-macrophage precursors (GMPs) and macrophage and DC precursors (MDPs) (Geissmann F et al. 2010). Another bone marrow precursor called the common monocyte progenitor (cMoP) was recently identified (Hettinger, Jan et al. “Origin of monocytes and macrophages in a committed progenitor.” Nature immunology vol. 14,8 (2013): 821-30. doi: 10.1038/ni.2638). cMoPs give rise to monocytes and their derivatives, but do not generate pDC or cDC. Phenotypically, cMoPs differ from MDPs in that they do not express CD135 (Hettinger J et al. 2013). Following development in the bone marrow, monocytes enter the peripheral blood stream, and around three days later, migrate to peripheral tissues, as a consequence of homeostasis and inflammation (Stefater, James A 3rd et al. “Metchnikoff's policemen: macrophages in development, homeostasis and regeneration.” Trends in molecular medicine vol. 17,12 (2011): 743-52.).

In tissues, monocytes differentiate into a range of tissue-specific macrophages (including TAM) or DC (monocyte-derived DCs) upon exposure to local growth factors such as pro-inflammatory cytokines and microbial compounds (Tacke, Frank, and Gwendalyn J Randolph. “Migratory fate and differentiation of blood monocyte subsets.” Immunobiology vol. 211,6-8 (2006): 609-18.). Monocytes Cultured with MCSF, IL-4, and TNF-α Yield D Cs and macrophages that closely resemble Inflammatory D Cs and Macrophages found In Vivo (see Sander, Jil et al. “Cellular Differentiation of Human Monocytes Is Regulated by Time-Dependent Interleukin-4 Signaling and the Transcriptional Regulator NCOR2.” Immunity vol. 47,6 (2017): 1051-1066.e12. doi: 10.1016/j.immuni.2017.11.024). Furthermore, it has been shown that IRF4 and MAFB are critical regulators of monocyte differentiation into mo-DCs and mo-Macs, respectively (see Goudot C, Coillard A, Villani A C, Gueguen P, Cros A, Sarkizova S, Tang-Huau T L, Bohec M, Baulande S, Hacohen N, Amigorena S, Segura E. Aryl Hydrocarbon Receptor Controls Monocyte Differentiation into Dendritic Cells versus Macrophages. Immunity. 2017 Sep. 19;47 (3): 582-596.e6).

Macrophage Colony-Stimulating Factor (M-CSF), encoded by the CSF1 gene, is a secreted molecule that promotes the proliferation, survival, and differentiation of mononuclear phagocytic cells such as monocytes and macrophages as well as bone marrow progenitor cells. M-CSF affects macrophages and monocytes in several ways, including stimulating increased phagocytic and chemotactic activity, and increased tumour cell cytotoxicity (Fixe P, Praloran V (June 1997). “Macrophage colony-stimulating-factor (M-CSF or CSF-1) and its receptor: structure-function relationships”. European Cytokine Network. 8 (2): 125-36).

Human peripheral blood monocytes can be defined by their expression of the cell surface markers CD14 (LPS co-receptor), CD16 (Fc gamma RIII), CD64 (Fc gamma RI) and the chemokine receptors CD192 (also known as CCR2) (a key mediator of monocyte migration) and CX3CR1 (fractalkine receptor) (Shi C and Pamer E G 2011). There are three different subsets of human monocytes: classical, intermediate and non-classical. These subpopulations can be further characterized by different levels of human leukocyte antigen-antigen D related (HLA-DR) (highest level on the intermediate population) and CD195 (also known as CCR5), as well as the receptors TNFR1 (CD120a) and TNFR2 (CD120b). TNFR1 expression is higher on intermediate monocytes, followed by classical and then non-classical monocytes. In contrast, TNFR2 is expressed higher on non-classical monocytes, followed by intermediate, with the lowest expression on the classical subpopulation (Hijdra, Danië11e et al. “Differential expression of TNFR1 (CD120a) and TNFR2 (CD120b) on subpopulations of human monocytes.” Journal of inflammation (London, England) vol. 9,1 38. 5 Oct. 2012).

In some embodiments, the myeloid cell as herein defined is a “classical monocyte”. Such “classical monocytes” exhibit phagocytic and microbial activity and have a low pro-inflammatory cytokine production. Typically, “classical” monocytes have the following phenotypes with regard to surface markers CD14^{hi}, CD16⁻, CD64⁺, CD62L⁺, TNFR1⁺ and TNER2^{low}. Such classical monocytes typically express the following chemokine receptors CD192^{hi} and CX3CR1^{low}.

In some embodiments, the myeloid cell as herein defined is an “intermediate monocyte”. Such intermediate monocytes actively produce TNF-α (in response to LPS), IL-1beta and IL-6. Typically, “intermediate” monocytes have the following phenotypes with regard to surface markers CD14^{hi}, CD16⁺, CD64⁺, HLA-DR^{hi}, TNFR1^{hi} and TNFR2⁺. Such classical monocytes typically express the following chemokine receptors CD192^{low} (CCR2^{low}) and CX3CR1^{hi}, and CD195⁺ (CCR5⁺).

In some embodiments, the myeloid cell as herein defined is an “non-classical monocyte”. Such “non-classical” monocytes are typically anti-inflammatory and actively produces IL-1RA. Typically, “non-classical” monocytes have the following phenotypes with regard to surface markers CD14^{low}, CD16^{hi}, CD64⁻, TNFR1^{low} and TNFR2^{hi}. Such classical monocytes typically express the following chemokine receptors CD192^{low} (CCR2^{low}) and CX3CR1^{hi}.

### 2.1.3 Macrophages

In some embodiments of the present application, the myeloid cell as herein defined is a macrophage. Macrophages are resident phagocytic cells in lymphoid and non-lymphoid tissues with highly diverse roles in the maintenance of an organism's biological integrity ranging from development, homeostasis, to repair, and immune responses to pathogens. Macrophages exert these functions through clearance of cell debris, production of growth factors, highly efficient phagocytosis (notably of tumor cells), and the production of inflammatory cytokines. Being equipped with a broad range of pathogen-recognition receptors they can act as sentinels and instantly respond to changes in physiology as well as challenges from outside. Based on specific programs of gene expression leading to the acquisition of different markers on the cellular surface, the secretion of certain cytokines as well as to metabolic adaptations, macrophages are usually classified into classically activated, pro-inflammatory or M1 macrophages, and alternatively activated, anti-inflammatory, or M2 macrophages. macrophages can be activated in a variety of ways. Direct macrophage activation is antigen-independent, relying on mechanisms such as pathogen associated molecular pattern recognition by Toll-like receptors (TLRs). Immune-complex mediated activation is antigen dependent but requires the presence of antigen-specific antibodies and absence of the inhibitory CD47-SIRPa interaction. Macrophages typically express the pan-surface marker CD68.

In some embodiments, the myeloid cell as herein defined in a pro-inflammatory M1 macrophage. Pro-inflammatory macrophages are typically induced by microbial products, such as the lipopolysaccharide (LPS) and other Toll-like receptors (TLRs) ligands, or by cytokines secreted by TH-1 lymphocytes, such as interferon gamma (IFN-γ) and tumor necrosis factor alpha (TNF-α). From the functional point of view, M1 macrophages are characterized by their ability to kill pathogens and present their antigens to T lymphocytes for initiation of adaptive responses. M1 macrophages typically expressed surface markers comprising CD80, CD86, CIITA, and MHC-II molecules; transcription factors comprising p65, STAT1, STAT3, IRF4 HIF1a and AP1, and metabolic enzymes comprising INOS, PFKFB3, PKM2, and ACOD1 and secretes cytokines comprising TNFα, IL1β, IL6, IL12 and IL23. In some embodiments, the myeloid cell as herein expresses one or more of these surface markers and metabolic enzymes and secretes one or more of these cytokines. In some embodiment, the myeloid cell is a M1 macrophages that express the surface markers and metabolic enzymes as above defined. Typically, such M1 macrophage secretes the cytokine as above mentioned.

In some embodiments, the myeloid cell as herein defined in an anti-inflammatory M2 macrophage.

M2a or anti-inflammatory macrophages (also named alternatively-activated macrophages) can be induced by IL-4 or IL-13 secreted by innate and adaptive immune cells, such as mast cells, basophils, and TH-2 lymphocytes. “Alternatively-activated” macrophages are characterized by an anti-inflammatory profile, which typically permits resolution of inflammation and tissue repair. M2a macrophages typically express surface markers comprising CD206, CD36, IL1Ra, and CD163; transcription factors comprising STAT6, GATA3, SOCS1 and PPARy; and metabolic enzymes comprising ARG1 and CARKL. M2a macrophages typically secretes cytokine comprising IL10 and TGFβ. In some embodiments, the myeloid cell as herein expresses one or more of these surface markers, transcription factors, and metabolic enzymes and secretes one or more of these cytokines. In some embodiment, the myeloid cell is a M2a macrophages that express the surface markers, transcription factors and metabolic enzymes as above defined. Typically, such M2a macrophage secretes the cytokine as above mentioned.

M2b or regulatory macrophages are typically induced by stimulation with immune complexes and TLR ligands or by IL-1R agonists. M2b macrophages regulate both immune and inflammatory reactions. M2b macrophages typically express surface markers comprising CD86 and MHC-II molecules; transcription factors comprising STAT3, IRF4 and p50 (NF-κβ); and metabolic enzymes comprising ARG1 and CARKL. M2a macrophages typically secretes cytokine comprising IL10 IL-1B, IL-6 and TNFα. In some embodiments, the myeloid cell as herein expresses one or more of these surface markers, transcription factors, and metabolic enzymes and secretes one or more of these cytokines. In some embodiment, the myeloid cell is a M2b macrophages that express the surface markers, transcription factors and metabolic enzymes as above defined. Typically, such M2b macrophage secretes the cytokine as above mentioned.

M2c macrophages are activated by glucocorticoids or IL-10 and exhibits a strong anti-inflammatory profile and a phagocytosis activity of apoptotic bodies. M2c macrophages typically express surface markers comprising CD163, TLR1 and TLR8; transcription factors comprising STAT3, STAT6, IRF4 and p50 (NF-κβ); and metabolic enzymes comprising ARG1 and GS. M2c macrophages typically secretes cytokine comprising IL10 and TGF-β. In some embodiments, the myeloid cell as herein expresses one or more of these surface markers, transcription factors, and metabolic enzymes and secretes one or more of these cytokines. In some embodiment, the myeloid cell is a M2c macrophages that express the surface markers, transcription factors and metabolic enzymes as above defined. Typically, such M2c macrophage secretes the cytokine as above mentioned.

M2d macrophages, also known as tumor-associated macrophages (TAMs), can be induced by TLR ligands and A2 adenosine receptor (A2R) agonists, or by IL-6; they contribute to tumor angiogenesis, growth and metastasis. M2d macrophages are activated by glucocorticoids or IL-10 and exhibits a strong anti-inflammatory profile and a phagocytosis activity of apoptotic bodies. M2d macrophages typically express surface markers comprising CD206, CD204 and CD163; transcription factors comprising STAT3, STAT1, IRF3 and p50 (NF-κβ); and metabolic enzymes comprising ARG1 and IDO. M2d macrophages typically secretes cytokine comprising IL10 and VEGF. In some embodiments, the myeloid cell as herein expresses one or more of these surface markers, transcription factors, and metabolic enzymes and secretes one or more of these cytokines. In some embodiment, the myeloid cell is a M2d macrophages that express the surface markers, transcription factors and metabolic enzymes as above defined. Typically, such M2d macrophage secretes the cytokine as above mentioned. TAM can also express CD11b, CD11c, CD64 and CD68 surface markers.

The expression of surface markers comprising CD14, CD206 and CD163 on M2 macrophages has been described to be associated with phagocytosis competence, in particular for cancer cells (Schulz, D., Severin, Y., Zanotelli, V. R. T. et al. In-Depth Characterization of Monocyte-Derived Macrophages using a Mass Cytometry-Based Phagocytosis Assay. Sci Rep 9, 1925 (2019). https://doi.org/10.1038/s41598-018-38127-9).

In some embodiments TAM can derive both from blood monocytes attracted by chemokines such as CCL2 or CSF-1, and from tissue-resident macrophages.

In some embodiments, the macrophage is a monocyte derived macrophage (MDMs). Monocyte-derived macrophages (MDMs), can be generated for example upon M-CSF treatment of monocytes.

### 2.1.4 Dendritic Cells

The Common dendritic cell precursor (CDP) can differentiate into plasmacytoid DC (pDC) or the preclassical (or conventional) DC (pre-cDC). Pre-cDC are the progenitors of the two major cDC subpopulations named cDC1 and cDC2. Precursors that can be used for in vitro generation of DCs can be extracted from the bone marrow. In the presence of GM-CSF, these precursors give rise to large number of cells that resemble tissue DC and are called bone marrow-derived dendritic cells (BMDC).

As per the present application, the myeloid cell can be a plasmacytoid DC (pDC) or a conventional DC (cDC, also known as myeloid dendritic cells). cDCs can be defined as MHCII+, CD11c⁺ CD123⁻. They are typically specialized at antigen uptake and presentation to naïve T cells, thus representing the “typical” antigen-presenting DC that primes adaptive immunity. cDCs have previously been subdivided into two subsets (cDC1 and cDC2) based on homology to murine equivalents (CD8a⁺/CD103⁺ and CD11b⁺ DCs respectively) and the differential expression of key transcription factors that drive their development; interferon regulatory factor (IRF) 8, basic leucine zipper transcriptional factor ATF-like 3 (BATF3) and DNA-binding protein inhibitor 2 (ID2) for cDC1 and IRF4, Neurogenic locus notch homolog protein 2 (Notch2) and Kruppel-like factor 4 (KLF4) for cDC2. In contrast, plasmacytoid DCs (pDCs) are CD11c⁻ CD123⁺ cells best characterized for their type I interferon (IFN-I) production during viral infection but can also perform a variety of other functions including T cell stimulation and pro-inflammatory cytokine and chemokine secretion. Other populations of peripheral blood DC (either distinct from or further subsets of cDCs and pDCs) have also been described based on the expression of various other markers including CD2, CD5, CD16, CD34, and Slan but have not been confirmed as distinct subsets by detailed transcriptomic or lineage analyses to date.

Dendritic cells also include tissular population such as epidermal dendritic cells, inflammatory dendritic epidermal cells, dermal lamina propria dendritic cells, intestinal dendritic cells and inflammatory dendritic cells.

In some embodiments, the cell is a cDC1 and typically expresses CD141, the chemokine receptor XCR1, C-type lectin CLEC9A, and the cell adhesion molecule CADM1. cDC1 can be for example generated in vitro from CD34+ progenitors after 21 days of culture with fms-like tyrosine kinase 3 ligand (Flt3L) and thrombopoietin (TPO) (Proietto A1, Mittag D, Roberts A W, Sprigg N, Wu L. The equivalents of human blood and spleen dendritic cell subtypes can be generated in vitro from human CD34+ stem cells in the presence of fms-like tyrosine kinase 3 ligand and thrombopoietin. Cell Mol Immunol. (2012) 9:446-54. doi: 10.1038/cmi.2012.48) or with Flt3L and murine bone marrow stromal cell lines (Lee J, Breton G, Aljoufi A, Zhou Y J, Puhr S, Nussenzweig MC, et al. Clonal analysis of human dendritic cell progenitor using a stromal cell culture. J Immunol Methods (2015) 425:21-6. doi: 10.1016/j.jim.2015.06.004). cDC1 have a central role in T-cell induction against tumors by presenting antigens and secreting IL-12, promoting CD4 Th1 and CD8 T-cell activity. cDC1 typically express the BDCA3, CD11c and CD103 surface markers.

In other embodiments, the dendritic cell is a cDC2 and typically expresses SIRPa (CD172a) and CD1c. cDC2 can typically be differentiated from CD34+ progenitors, after 21 days of culture with Flt3L and TPO or with Flt3L and murine bone marrow stromal cell lines (se references above). cCD2 typically express the BDCA1, CD11b, and CD11c surface markers.

In other embodiments, the dendritic cell is a plasmacytoid DC (pDC). pDC arise directly from the CDP. Plasmacytoid D Cs are present in the bone marrow and all peripheral organs. They are relatively long-lived and display a characteristic surface phenotype and morphology, including a highly developed secretory compartment. pDCs are specialized to respond to viral infection with a massive production of type I interferons (IFNs). They can also act as antigen-presenting cells and control T cell responses. Typically, this cell is characterized by the absence of expression of CD11c and the expression of CD123, CD303, and CD304. Typically, pDC cells are characterized by the secretion of high levels of IFN-α/β (generally upon TLR7/9 stimulation).

In some embodiments, the dendritic cell is a monocyte-derived DC (mo-DC). Such cell can be derived from monocytes for example upon stimulation with GM-CSF (Granulocyte-macrophage colony-stimulating also known as colony-stimulating factor 2, CSF2) and IL-4. Typically, mo-DC differentiation depends on IRF4 expression. Mo-DC are potent T cell stimulatory cells. Mo-DCs typically express CD11b and CD11c as well as MHCII surface markers.

In some embodiments, the dendritic cell is characterized by expression of CD14, CD1c, SIRPa, CD20+, and FcERI.

In some embodiments, the dendritic cell is a CD103⁺ cell, which is typically derived from pre-cDCs. In other embodiments, the dendritic cell is a CX3CR1⁺ DC which is typically derived from Ly6C⁺ monocytes.

In some embodiments, the dendritic cells as used herein are mature DCs. Mature DCs have the ability to activate antigen-specific naïve T cells in secondary lymphoid organs. Typically, mature dendritic cells express high levels of MHC-II molecules. In some embodiments, mature DCs express the chemokine receptor CCR7. Mature DCs exhibit typically a decreased endocytic activity and an increased secretion of cytokines essential for T-cell activation.

In some embodiments, DCs are immature DCs. Immature DCs are poor inducers of naïve lymphocyte effector responses, since they have low surface expression of co-stimulatory molecules, low expression of chemokine receptors (CCR7), and do not release immunostimulatory cytokines. These “immature” cells, though, are very efficient in antigen capture due to their high endocytic capacity, via receptor-mediated endocytosis (including lectin-; Toll-like-, FC-and complement receptors) and macropinocytosis.

### 2.1.5 Granulocytes

Granulocytes are at all ages the most abundant type of myeloid cells in the blood stream and can be further subdivided into neutrophils, eosinophils, and basophils. GM-CSF (Granulocyte-macrophage colony-stimulating also known as colony-stimulating factor 2, CSF2) factor and G-CSF (Granulocyte colony-stimulating factor), the cytokines that drive differentiation of precursors into granulocytes and promote the survival of mature neutrophils

In adults, neutrophils are the most frequent granulocytes. They are constantly generated in a high number in the BM and circulate with the blood stream until activated by signals that are provoked by resident macrophages at the site of infection or injury. Once in the tissue neutrophils combat microorganism via phagocytosis, the release of microbicidal proteins and by neutrophil extracellular trap formation.

In the adult mammal, neutrophils are produced in the bone marrow and released at a steady rate under homeostatic conditions. Differentiation from hematopoietic stem cells to common myeloid progenitor cells to lineage-committed progenitors that mature into neutrophils takes more than 10 days. Several transcription factors-including PU.1, CCAAT/enhancer binding protein a (C/EBPa), growth factor independence 1 (GFI1), and C/EBPE—are necessary for neutrophil maturation during steady-state granulopoiesis. Various unipotent neutrophils progenitors have been identified in bone marrow such as preNeu that is CD66b⁺, CD117⁻, CD34⁻; or a heterogeneous early neutrophil progenitor (hNeP) that is CD66b⁺, CD117⁺ and can be fractionated into CD34⁺ and CD34⁻ subsets.

Human bone marrow neutrophils exposed to GM-CSF can differentiate into neutrophil-DC hybrids, exhibiting a DC-like phenotype and antigen-presenting function, while still maintaining several neutrophil features. To execute a rapid and precise response to infections, neutrophils rely on preformed molecules stored in a variety of intracellular granules. Granule proteins regulate adhesion, transmigration, phagocytosis, and neutrophil extracellular trap (NET) formation. The secretory proteins also constitute some of the most toxic, readily releasable factors produced by the human body. Thus, neutrophil degranulation, although important for controlling infections, can induce potent proinflammatory responses (for review regarding neutrophils see Ley, Klaus et al. “Neutrophils: New insights and open questions.” Science immunology vol. 3,30 (2018): eaat4579. doi: 10.1126/sciimmunol.aat4579).

Eosinophils are resident in various organs such as the gastrointestinal tract and BM and contribute to tissue and immune homeostasis. Only a minor part of the eosinophils circulates in the peripheral blood and is recruited mainly upon TH2 responses into sites of inflammation. Within the tissues they produce several cytokines and lipid mediators and release toxic granule proteins. Eosinophils are associated with immune responses directed against parasites or allergens and contribute to immune pathology and parasite clearance. The cytokines IL-3, IL-5, and GM-CSF are especially important for eosinophil expansion. Of these three cytokines, IL-5 is the most specific to the eosinophil lineage and is responsible for selective differentiation of eosinophils.

Basophils are the least common granulocytes in the circulation. They play a central role in inflammatory and immediate allergic reactions. They are able to release potent inflammatory mediators, such as histamine, proteases, chemotactic factors, cytokines, and metabolites of arachidonic acid that act on the vasculature, smooth muscle, connective tissue, mucous glands, and inflammatory cells.

### 2.2 Sources of Cells and Expansion Thereof

### 2.2.1 Sources

As per the present application, the myeloid cell is selected from a dendritic cell, a macrophage, or monocyte or a granulocyte including their progenitors (typically multipotent progenitors) and progeny as previously defined. Well-suited myeloid cells as per the present invention are phagocytic cells, in particular neutrophils, monocytes, macrophages, and dendritic cells. In some embodiments the myeloid cell has phagocytic, targeted cellular cytotoxicity, antigen presentation, and/or cytokine secretion targeted activity.

The cells typically are primary cells, such as those isolated directly from a subject and/or isolated from a subject and frozen. In other embodiments, the cells are derived from cell lines. The cells can also be obtained from a xenogeneic source, such as a mouse, a rat, a non-human primate, or a pig. Preferably, the cells are human cells. In some embodiments, the myeloid cell as herein disclosed, such as monocyte, dendritic cells (including mo-DCs) or macrophages (including monocyte-derived macrophages) can be obtained from human induced pluripotent stem cells (IPSCs) (Hansen, Marten et al. “Efficient production of erythroid, megakaryocytic and myeloid cells, using single cell-derived iPSC colony differentiation.” Stem cell research vol. 29 (2018): 232-244; Lachmann, Nico et al. “Large-scale hematopoietic differentiation of human induced pluripotent stem cells provides granulocytes or macrophages for cell replacement therapies.” Stem cell reports vol. 4,2 (2015): 282-96. doi: 10.1016/j.stemcr.2015.01.005; Mukherjee C., Hale C., Mukhopadhyay S. (2018) A Simple Multistep Protocol for Differentiating Human Induced Pluripotent Stem Cells into Functional Macrophages. In: Rousselet G. (eds) Macrophages. Methods in Molecular Biology, vol 1784. Humana Press, New York, NY., Sachamitr, Patty et al. “Directed Differentiation of Human Induced Pluripotent Stem Cells into Dendritic Cells Displaying Tolerogenic Properties and Resembling the CD141+ Subset.” Frontiers in immunology vol. 8 1935. 8 Jan. 2018, doi: 10.3389/fimmu.2017.01935; Hiramatsu, N., Yamamoto, N., Isogai, S. et al. An analysis of monocytes and dendritic cells differentiated from human peripheral blood monocyte-derived induced pluripotent stem cells. Med Mol Morphol 53, 63-72 (2020).

With reference to the subject to be treated, the cells of the invention may be allogeneic and/or autologous. The cells and compositions containing the cells myeloid cells according to the invention are typically isolated from a sample, notably a biological sample, e.g., obtained from or derived from a subject. Typically, the subject is in need for a cell therapy (adoptive cell therapy) and/or is the one who will receive the cell therapy. The subject is preferably a mammal, notably a human. In one embodiment of the invention, the subject has a cancer.

In autologous immune cell therapy, immune cells (i.e., immunoresponsive myeloid cells as herein described) are collected from the patient, modified as described herein, and returned to the patient. In allogeneic immune cell therapy, immune cells are collected from healthy donors, rather than the patient, modified as described herein, and administered to patients. Typically, these are HLA matched to reduce the likelihood of rejection by the host. The myeloid cells as herein described may thus also comprise modifications such as disruption or removal of HLA class I molecules. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression.

The samples include tissue, fluid, and other samples taken directly from the subject, as well as samples resulting from one or more processing steps, such as separation, centrifugation, genetic engineering (for example transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. Preferably, the sample from which the cells are derived or isolated is blood or a blood-derived sample, or is or is derived from an apheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, and/or cells derived therefrom. Samples include, in the context of cell therapy (typically adoptive cell therapy) samples from autologous and allogeneic sources.

In certain embodiments, the cells can be obtained from a unit of blood collected from a subject using any number of techniques known to the skilled artisan, such as Ficoll separation. In one embodiment, cells from the circulating blood of an individual can be obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media. In another embodiment, cells can be isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of myeloid cells, typically the monocytes, macrophages and/or dendritic cells can be further isolated by positive or negative selection techniques.

In some embodiments, mononuclear cells typically isolated as above described can be depleted of cells expressing certain antigens, including, but not limited to, CD34, CD3, CD4, CD8, CD14, CD19 or CD20. Depletion of these cells can be accomplished using an isolated antibody, a biological sample comprising an antibody, such as ascites fluid, an antibody bound to a physical support, and a cell bound antibody.

Enrichment of a specific myeloid cell subpopulation, typically granulocyte, monocyte, macrophage and/or dendritic cell population by negative or positive selection can be accomplished using a combination of antibodies directed to surface markers unique to the negatively or positively selected cells. A preferred method is cell sorting and/or selection via magnetic immunoadherence or flow cytometry that uses a cocktail of monoclonal antibodies directed to cell surface markers present on the cells negatively or positively selected. For example, enrich of a cell population for monocytes, macrophages and/or dendritic cells by negative selection can be accomplished using a monoclonal antibody cocktail that typically includes antibodies to CD34, CD3, CD4, CD8, CD14, CD19 or CD20 or any against one or more of the surface markers as previously defined for the various subpopulations of myeloid cells. Thus, identification of the phenotype of myeloid cells are herein defined, i.e., expressing specific markers can be performed using flow cytometry analysis after staining with antibodies against selected phenotype markers. In some embodiment analysis can be performed at least 6 h notably at least 12 h after stimulation. Corresponding isotype can be used as a negative control. In some embodiments, detection of cytokine secretion can be achieved by collecting cell-supernatants and performing ELISA assays.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. The use of high concentrations of cells can result in increased cell yield, cell activation, and cell expansion.

In some embodiment, a population of cells comprises the monocytes, macrophages, granulocytes or dendritic cells of the present invention. Examples of a population of cells include, but are not limited to, peripheral blood mononuclear cells, cord blood cells, a purified population of monocytes, macrophages, granulocytes, or dendritic cells, and a cell line. In another embodiment, peripheral blood mononuclear cells comprise the population of monocytes, macrophages, granulocytes, or dendritic cells. In yet another embodiment, purified cells comprise the population of monocytes, macrophages, granulocytes or dendritic cells.

In some embodiments, the myeloid cell according to the present application has one or more upregulated M1 markers and one or more downregulated M2 markers as previously detailed. For example, the myeloid cell has at least one M1 marker, such as HLA DR, CD86, CD80, and/or PDL1, which is upregulated. In some embodiments, the myeloid cell has at least one M2 marker, such as CD206 and/or CD163, which is downregulated. In one embodiment, the cell has at least one upregulated M1 marker and at least one downregulated M2 marker. In some embodiments, the cell has a M1 phenotype as previously defined.

### 2.2.2 Cell Expansion

In one embodiment, the cells or population of cells comprising monocytes, macrophages, or dendritic cells are cultured for expansion. In another embodiment, the cells or population of cells comprising progenitor cells (including also IPSCs) are cultured for differentiation and expansion into monocytes, macrophages, or dendritic cells. The present invention also comprises expanding a population of monocytes, macrophages, or dendritic cells comprising a chimeric antigen receptor as described herein.

As demonstrated by the data disclosed herein, expanding the cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the cells expand in the range of about 20 fold to about 50 fold.

Following culturing, the cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The culture medium may be replaced during the culture of the cells at any time. Preferably, the culture medium is replaced about every 2 to 3 days. The cells are then harvested from the culture apparatus whereupon the cells can be used immediately or stored for use at a later time

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

In one embodiment, the cells may be cultured for several hours (about 3 hours) to about 14 days or any hourly integer value in between. Conditions appropriate for cell culture include an appropriate media (e.g., macrophage complete medium, DMEM/F12, DMEM/F 12-10 (Invitrogen)) that may contain factors necessary for proliferation and viability, including serum (e.g., fetal bovine or human serum), L-glutamine, insulin, M-CSF, GM-CSF, IL-10, IL-12, IL-15, TGF-β, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of the cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

The medium used to culture the cells may include an agent that can activate the cells. For example, an agent that is known in the art to activate the monocyte, macrophage or dendritic cell is included in the culture medium, including but not limited to M-CSF, GM-CSF, G-CSF, IL-3, IL-4, IL-5, IL-10, IL-12, IL-15, TGF-β, TNF-α, IFNγ, TLR ligands and combination thereof. Exemplary combinations of cytokines and transcription factors involved in myeloid cell differentiation and maturation have been described previously. Differentiation of peripheral blood monocytes in vitro into macrophages can be achieved for example by following the guidelines proposed by Murray, P. J. et al. Macrophage Activation and Polarization: Nomenclature and Experimental Guidelines. Immunity 41, 14-20 (2014). Approaches to obtain cDCs are for example illustrated in F Sallusto, A Lanzavecchia; “Efficient presentation of soluble antigen by cultured human dendritic cells is maintained by granulocyte/macrophage colony-stimulating factor plus interleukin 4 and downregulated by tumor necrosis factor alpha”. J Exp Med 1 Apr. 1994; 179 (4): 1109-1118. Generation of dendritic cells from CD34+ cells culture is also described in Balan, Sreekumar et al. “Large-Scale Human Dendritic Cell Differentiation Revealing Notch-Dependent Lineage Bifurcation and Heterogeneity.” Cell reports vol. 24,7 (2018): 1902-1915.e6. Existing approaches to obtain cDCs from various cell subsets are also summarized in table 1 from Calmeiro, João et al. “Dendritic Cell Vaccines for Cancer Immunotherapy: The Role of Human Conventional Type 1 Dendritic Cells.” Pharmaceutics vol. 12,2 158. 15 Feb. 2020, but see also Sander, Jil et al. “Cellular Differentiation of Human Monocytes Is Regulated by Time-Dependent Interleukin-4 Signaling and the Transcriptional Regulator NCOR2.” Immunity vol. 47,6 (2017): 1051-1066.e12. doi: 10.1016/j.immuni.2017.11.024 and Goudot C, Coillard A, Villani A C, Gueguen P, Cros A, Sarkizova S, Tang-Huau T L, Bohec M, Baulande S, Hacohen N, Amigorena S, Segura E. Aryl Hydrocarbon Receptor Controls Monocyte Differentiation into Dendritic Cells versus Macrophages. Immunity. 2017 Sep. 19;47 (3): 582-596.e6.

In some embodiments, the myeloid cell as herein disclosed, such as monocyte, dendritic cells (including mo-DCs) or macrophages (including monocyte-derived macrophages) can be obtained from human induced pluripotent stem cells (IPSCs) (Hansen, Marten et al. “Efficient production of erythroid, megakaryocytic and myeloid cells, using single cell-derived iPSC colony differentiation.” Stem cell research vol. 29 (2018): 232-244; Lachmann, Nico et al. “Large-scale hematopoietic differentiation of human induced pluripotent stem cells provides granulocytes or macrophages for cell replacement therapies.” Stem cell reports vol. 4,2 (2015): 282-96. doi: 10.1016/j.stemcr.2015.01.005; Mukherjee C., Hale C., Mukhopadhyay S. (2018) A Simple Multistep Protocol for Differentiating Human Induced Pluripotent Stem Cells into Functional Macrophages. In: Rousselet G. (eds) Macrophages. Methods in Molecular Biology, vol 1784. Humana Press, New York, NY., Sachamitr, Patty et al. “Directed Differentiation of Human Induced Pluripotent Stem Cells into Dendritic Cells Displaying Tolerogenic Properties and Resembling the CD141+ Subset.” Frontiers in immunology vol. 8 1935. 8 Jan. 2018, doi: 10.3389/fimmu.2017.01935; Hiramatsu, N., Yamamoto, N., Isogai, S. et al. An analysis of monocytes and dendritic cells differentiated from human peripheral blood monocyte-derived induced pluripotent stem cells. Med Mol Morphol 53, 63-72 (2020).

### 2.3 Cell Engineering

### 2.3.1 Cells Overexpressing Bcl2

Myeloid cells according to the present invention are modified to express or to overexpress bcl2.

Bcl-2 (B-cell lymphoma 2), is encoded in humans by the BCL2 gene (located on chromosome 18:63,123,346-63,320,128), is an integral outer mitochondrial membrane protein, founding member of the Bcl-2 family of regulator proteins that regulate cell death (apoptosis), by either inhibiting (anti-apoptotic) or inducing (pro-apoptotic) apoptosis. Bcl-2 derives its name from B-cell lymphoma 2, as it is the second member of a range of proteins initially described in chromosomal translocations involving chromosomes 14 and 18 in follicular lymphomas. BCL2 protein has several isoforms. One exemplary protein sequence is SEQ ID NO:1 (BLC2 protein, UniProtKB P10415 (BCL2_HUMAN)), canonical isoform alpha), as follows: >sp|P10415|BCL2_HUMAN Apoptosis regulator Bcl-2 OS═Homo sapiens OX=9606 GN=BCL2 PE=1 SV=2

MAHAGRTGYDNREIVMKYIHYKLSQRGYEWDAGDVGAAPPGAAPAPGI
FSSQPGHTPHPAASRDPVARTSPLQTPAAPGAAAGPALSPVPPVVHLT
LRQAGDDFSRRYRRDFAEMSSQLHLTPFTARGRFATVVEELFRDGVNW
GRIVAFFEFGGVMCVESVNREMSPLVDNIALWMTEYLNRHLHTWIQDN
GGWDAFVELYGPSMRPLFDFSWLSLKTLLSLALVGACITLGAYLGHK

The present invention also encompasses all BLC2 isoforms such as notably isoform beta of SEQ ID NO:2 (isoform beta, identifier: P10415-2): >sp|P10415-2|BCL2_HUMAN Isoform Beta of Apoptosis regulator Bcl-2 OS═Homo sapiensOX=9606GN=BCL2

MAHAGRTGYDNREIVMKYIHYKLSQRGYEWDAGDVGAAPPGAAPAPGI
FSSQPGHTPHPAASRDPVARTSPLQTPAAPGAAAGPALSPVPPVVHLT
LRQAGDDFSRRYRRDFAEMSSQLHLTPFTARGRFATVVEELFRDGVNW
GRIVAFFEFGGVMCVESVNREMSPLVDNIALWMTEYLNRHLHTWIQDN
GGWVGALGDVSLG

Typically, the present invention encompasses the proteins encoded by the human bc/2 gene located on chromosome 18:63,313,802-63,318,812 reverse strand (referenced in in databases HGNC: 990 NCBI Entrez Gene: 596 Ensembl: ENSG00000171791).

It is however understood that polymorphisms or variants with different sequences exist in various subject genomes. The term BCL2 according to the invention thus encompasses all mammalian variants of BCL2, and genes that encode this protein with at least 75%, 80%, or typically 85%, 90%, or 95% identical to SEQ ID NO: 1 or 2 and that has BCL2 activity. BCL2 activity can be assessed for example by inducing apoptosis in vitro (for example using low dose Aphidicolin (0.03 nM) or infection by poxviruses) and measuring the resistance provided by addition of BCL2.

As used herein, the expression “percentage of identity” between two sequences, means the percentage of identical bases or amino acids between the two sequences to be compared, obtained with the best alignment of said sequences, this percentage being purely statistical and the differences between these two sequences being randomly spread over the two sequences. As used herein, “best alignment” or “optimal alignment”, means the alignment for which the determined percentage of identity (see below) is the highest. Sequence comparison between two nucleic acids sequences is usually realized by comparing these sequences that have been previously aligned according to the best alignment; this comparison is realized on segments of comparison in order to identify and compared the local regions of similarity. The best sequences alignment to perform comparison can be realized, besides manually, by using the global homology algorithm developed by SMITH and WATERMAN (Ad. App. Math., vol. 2, p: 482, 1981), by using the local homology algorithm developed by NEDDLEMAN and WUNSCH (J. Mol. Biol, vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, WI USA), by using the MUSCLE multiple alignment algorithms (Edgar, Robert C, Nucleic Acids Research, vol. 32, p: 1792, 2004). To get the best local alignment, one can preferably use BLAST software. The identity percentage between two sequences is determined by comparing these two sequences optimally aligned, the sequences being able to comprise additions or deletions in respect to the reference sequence in order to get the optimal alignment between these two sequences. The percentage of identity is calculated by determining the number of identical positions between these two sequences, and dividing this number by the total number of compared positions, and by multiplying the result obtained by 100 to get the percentage of identity between these two sequences.

The term “expressing a polynucleotide” means when a polynucleotide is transcribed to mRNA and the mRNA is translated to a polypeptide. The term “overexpress” generally refers to any amount greater than or equal to an expression level exhibited by a reference standard (typically the corresponding non genetically modified cell). The terms “increased expression”, “overexpress,” “overexpressing,” “overexpressed” and “overexpression” in the present invention refer an expression of a gene product or a polypeptide at a level greater than the expression of the same gene product or polypeptide prior to a genetic alteration of the host cell or in a comparable host which has not been genetically altered at defined conditions. By “increased expression”, or “overexpression” it is meant herein that the expression is increased of at least 30%, 40%, 50%, 60%, 70%, 80%, 90%, 95% or 99% or more as compared to normal levels in the corresponding non genetically modified cell. If the said myeloid cell does not comprise the given gene product (i.e;, the bcl2 gene), it is possible to introduce it into the said cell for expression; in this case, any detectable expression is encompassed by the term “overexpression.” Typically, BCL2 can be expressed in in a single polypeptide in fusion with a protein of interest (such as a GFP marker or a chimeric antigen protein) separated by 2A peptides. Thus BCL2 transgene expression in modified cells can be detected by way of measuring GFP or any other protein of interest such as the CAR. Alternatively, anti-BCL2 antibodies can be used to detect expression of BLC2.

According to the present invention the modified, or genetically engineered, myeloid cell according to the present invention exhibits an overexpression, or an increased expression of the BCL2 protein. In some particular embodiments, the modified myeloid cell is a modified macrophage. The modified macrophage is derived from any macrophage according to the present disclosure such as for example a monocyte-derived macrophage.

In some embodiments, targeted effector activity of a phagocytic myeloid cell can be enhanced by inhibition of either CD47 or SIRPa activity.

CD47 and/or SIRPa activity may be inhibited by treating the phagocytic cell with an anti-CD47 or anti-SIRPa antibody.

Alternatively, CD47 or SIRPa activity may be inhibited by any method known to those skilled in the art. For example, the myeloid cell may be genetically engineered such that the cell has a reduced expression of the SIRPa protein or expresses a non-functional SIRPa protein.

“Reduced expression” as per the invention refers to a decrease of protein expression of at least 30%, 40%, 50%, 60%, 70%, 80%, 90%, 95% or 99% or more as compared to normal levels in the corresponding non-modified cell.

By “non-functional” protein it is herein intended a protein with a reduced activity or a lack of detectable activity as described above. The expression “reduced activity” as used herein refers to a decrease of activity of at least 30%, 40%, 50%, 60%, 70%, 80%, 90%, 95% or 99% or more as compared to the activity or level of the protein which is not inhibited. Preferentially, the inhibition activity leads to the absence in the cell of substantial detectable activity of the given protein.

In some embodiments, the modified cell described herein has the capacity to deliver an agent, a biological agent or a therapeutic agent to the target cell. The cell may be modified or engineered to deliver an agent to a target, wherein the agent is selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate or the like, a lipid, a hormone, a microsome, a derivative or a variation thereof, and any combination thereof. As a non-limiting example, a macrophage modified with a CAR that targets a tumor antigen is capable of secreting an agent, such as a cytokine or antibody, to aid in macrophage function. Antibodies, such as anti-CD47/anti-SIRPa mAb, may also aid in macrophage function. In yet another example, the macrophage modified with a CAR that targets a tumor antigen is engineered to encode a siRNA that aids macrophage function by downregulating inhibitory genes (i.e. SIRPa). Another example, the CAR macrophage is engineered to express a dominant negative (or otherwise mutated) version of a receptor or enzyme that aids in macrophage function.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### 2.3.2 Cells Expressing an Antigen Recognizing Receptor

In some embodiments, the myeloid cells express one or more antigen-recognizing receptors on the surface. The cells thus may comprise one or more nucleic acids that encode one or more antigen-specific receptors, optionally operably linked to a heterologous regulatory control sequence. Typically, such antigen-specific receptors bind the target antigen with a K_{d} binding affinity of 10-6M or less, 10-7 M or less, 10-8 M or less, 10-9 M or less, 10-10 M or less, or 10-11 M or less (lower numbers indicating greater binding affinity).

Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types. The nucleic acids and their regulatory control sequences are typically heterologous. For example, the nucleic acid encoding the antigen-specific receptor may be heterologous to the immune cell and operatively linked to an endogenous promoter of the T-cell receptor such that its expression is under control of the endogenous promoter.

Among the antigen-specific receptors as per the invention are chimeric antigen receptors (CAR).

In some embodiments, the engineered antigen-specific receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5 (215) (December 2013)).

Chimeric antigen receptors (CARs), (also known as Chimeric immunoreceptors, Chimeric T cell receptors, Artificial T cell receptors) are engineered antigen-specific receptors, which graft an arbitrary specificity onto an immune effector cell. Typically, these receptors are used to graft the specificity of a monoclonal antibody onto an immune cell (e.g. an immunoresponsive cell as defined herein), with transfer of their coding sequence facilitated by viral vectors (typically retroviral vector).

CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone. The CAR may include
(a) an extracellular antigen-binding domain,
(b) a transmembrane domain,
(c) optionally a co-stimulatory domain, and
(d) an intracellular signaling domain.

In some embodiments, the CAR is constructed with a specificity for a particular antigen (or marker or ligand), such as an antigen expressed in a particular cell type to be targeted by adoptive cell therapy, such as a cancer marker. The CAR typically includes in its extracellular portion one or more antigen binding molecules, such as one or more antigen-binding fragment, domain, or portion of an antibody, typically one or more antibody variable domains. For example, the extracellular antigen-binding domain may comprise a light chain variable domain and a heavy chain variable domain, typically as an scFv.

The moieties used to bind to antigen include three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3 (4): 388-398 (see notably table 1) and are included in the present application.

Antibodies include chimeric, humanized or human antibodies, and can be further affinity matured and selected as described above. Chimeric or humanized scFv's derived from rodent immunoglobulins (e.g. mice, rat) are commonly used, as they are easily derived from well-characterized monoclonal antibodies. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

Typically, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR comprises an antibody heavy chain variable domain that specifically binds the antigen, such as a cancer marker or cell surface antigen of a cell or disease to be targeted, such as a tumor cell or a cancer cell, such as any of the target antigens described herein or known in the art.

In some embodiments, the CAR contains an antibody or an antigen-binding fragment (e.g. scFv) that specifically recognizes an antigen, such as an intact antigen, expressed on the surface of a cell.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen-specific receptor. Among the antigen-specific receptors are functional non-TCR antigen-specific receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that is naturally associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain can be derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR), the y subunit of Fc receptor. The transmembrane domain can also be synthetic. In some embodiments, the transmembrane domain is derived from CD28, CD8, CD3-zeta, or the y subunit of Fc receptor.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3 Z-chain, which is the primary transmitter of signals from endogenous TCRs. Second generation CARs typically further comprise intracellular signaling domains from various costimulatory protein receptors (e.g., CD28, 41BB (CD28), ICOS) to the cytoplasmic tail of the CAR to provide additional signals to the T cell. Co-stimulatory domains include domains derived from human CD28, 4-1BB (CD137), ICOS-1, CD27, OX 40 (CD137), DAP10, and GITR (AITR). Combinations of two co-stimulatory domains are contemplated, e.g. CD28 and 4-1BB, or CD28 and OX40. Third generation CARs combine multiple signaling domains, such as CD3z-CD28-4-1BB or CD3z-CD28-OX40, to augment potency.

The intracellular signaling domain can be from an intracellular component of the TCR complex, such as a TCR CD3+ chain that mediates T-cell activation and cytotoxicity, e.g., the CD3 zeta chain. Alternative well-suited intracellular signaling domains include the intracellular component of various proteins including but to limited to CD3, the y subunit of Fc receptor (such as of FcaRly), CD64, CD32, CD32b, CD32c, CCD16, CD16a, CD16b, MEGF10, CD40, the Toll-like receptor/Interleukin (IL)-1 receptor (TLR/IL-1R) superfamily, members of the BAI family of phosphatidylserine receptor, such as BAI1, or members from the TAM family of phosphatidylserine receptors, such as MerTK (Penberthy, Kristen K, and Kodi S Ravichandran. “Apoptotic cell recognition receptors and scavenger receptors.” Immunological reviews vol. 269, 1 (2016): 44-59, but see also Morrissey M A, Williamson A P, Steinbach A M, Roberts E W, Kern N, Headley M B, Vale R D. Chimeric antigen receptors that trigger phagocytosis. Elife. 2018 Jun. 4;7: e36688), and/or other CD transmembrane domains. The CAR can also further include a portion of one or more additional molecules such as Fc receptor y, CD8, CD4, CD25, CD16. Typically, TLR signaling domains include the Toll/interleukin receptor homology domain, TIR as well as any intracellular domain interacting with MyDDosome and/or TRIFosome clusters such as in particular with MyD88, TIRAP, TRIF and/or TRAM).

The intracellular signaling domain may also or alternatively comprise a modified CD3 zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The intracellular signaling region of CD3-zeta is residues 22-164 of SEQ ID NO: 4. ITAM1 is located around amino acid residues 61-89, ITAM2 around amino acid residues 100-128, and ITAM3 around residues 131-159. Thus, the modified CD3 zeta polypeptide may have any one of ITAM1, ITAM2, or ITAM3 inactivated. Alternatively, the modified CD3 zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3 zeta polypeptide retains only ITAM1 and the remaining CD37 domain is deleted (residues 90-164). As another example, ITAM1 is substituted with the amino acid sequence of ITAM3, and the remaining CD37 domain is deleted (residues 90-164). See, for example, Bridgeman et al., Clin. Exp. Immunol. 175 (2): 258-67 (2014); Zhao et al., J. Immunol. 183 (9): 5563-74 (2009); Maus et al., WO 2018/132506; Sadelain et al., WO/2019/133969, Feucht et al., Nat Med. 25 (1): 82-88 (2019).

Thus, in some aspects, the antigen binding molecule is linked to one or more cell signaling modules including but to limited to CD3 (in particular CD247, CD3z) and/or modified CD3 (notably modified CD247 or CD3z), the y subunit of Fc receptor (such as of FcaRly), CD64, CD32, CD32b, CD32c, CD16, CD16a, CD16b, MEGF10, CD40, the Toll-like receptor/Interleukin (IL)-1 receptor (TLR/IL-1R) superfamily, members of the BAI family of phosphatidylserine receptor, such as BAI1, or members from the TAM family of phosphatidylserine receptors, such as MerTK, and/or other CD transmembrane domains. The CAR can also further include a portion of one or more additional molecules such as Fc receptor y, CD8, CD4, CD25, CD16. Typically, TLR signaling domains include the Toll/interleukin receptor homology domain, TIR as well as any intracellular domain interacting with MyDDosome and/or TRIFosome clusters such as in particular with MyD88, TIRAP, TRIF and/or TRAM). These one or more signaling domains may be combined with one or more co-stimulatory domains include domains derived, for example, from human CD28, 4-1BB (CD137), ICOS-1, CD27, OX 40 (CD137), DAP10, GITR (AITR), CD80, CD86, CD40, CD16, CD32 and CD64.

In some embodiments, upon ligation of the CAR, the cytoplasmic domain or intracellular signaling domain of the CAR activates at least one of the normal effector functions or responses of the corresponding non-engineered immune cell (typically a phagocytic cell such as a macrophage, a dendritic cell, a monocyte or a granulocyte). For example, the CAR can induce a function of a macrophage, a dendritic cell or a monocyte such as phagocytic activity, cytotoxic activity, or secretion of cytokines or other factors.

In some embodiments, the intracellular signaling domain(s) include the cytoplasmic sequences of the T cell receptor (TCR), and in some aspects also those of co-receptors that in the natural context act in concert with such receptor to initiate signal transduction following antigen-specific receptor engagement, and/or a variant of such molecules, and/or any synthetic sequence that has the same functional capability.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR adapted for myeloid cells according to the present invention can include one or both of such signaling components.

In some aspects, the CAR includes a primary cytoplasmic signaling sequence that regulates primary activation of the FcR or the Toll-like receptor or any one of CD40, CD64, CD32, CD32b, CD32c, CD16a, CD16bn CD16c, members of the BAI family of phosphatidylserine receptor, such as BAI1, or members from the TAM family of phosphatidylserine receptors, such as MerTK, either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, CD66d, and Jedi-1, and Megf10. In some embodiments, cytoplasmic signaling molecule(s) in the CAR contain(s) a cytoplasmic signaling domain, portion thereof, or sequence derived from CD3 zeta, Jedi-1, or Megf10.

The CAR can also include a signaling domain and/or transmembrane portion of a costimulatory receptor, such as CD80, CD86, CD40, CD16, CD32 and CD64. In some aspects, the same CAR includes both the activating and costimulatory components; alternatively, the activating domain is provided by one CAR whereas the costimulatory component is provided by another CAR recognizing another antigen.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

### 2.4 Antigens

Among the antigens targeted by the antigen-specific receptors are those expressed in the context of a disease, condition, or cell type to be targeted via the adoptive cell therapy. Among the diseases and conditions are proliferative, neoplastic, and malignant diseases and disorders, more particularly cancers. Infectious diseases and autoimmune, inflammatory or allergic diseases are also contemplated.

The cancer may be a “solid cancer” or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the invention is a cancer affecting the blood, bone marrow and lymphoid system as described above. In some embodiments, the cancer is, or is associated, with multiple myeloma.

Diseases according to the invention also encompass infectious diseases or conditions, such as, but not limited to, viral, retroviral, bacterial, and protozoal infections, HIV immunodeficiency, Cytomegalovirus (CMV), Epstein-Barr virus (EBV), adenovirus, BK polyomavirus.

Diseases according to the invention also encompass autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1B1 (CYP1B), HER2/neu, p95HER2, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI). Peptide epitopes of tumor antigens, including universal tumor antigens, are known in the art and, in some aspects, can be used to generate MHC-restricted antigen-specific receptors, such as TCRs or TCR-like CARs (see e.g. published PCT application No. WO2011009173 or WO2012135854 and published U.S. application No. US20140065708).

In some aspects, the antigen is expressed on multiple myeloma, such as CD38, CD138, and/or CS-1. Other exemplary multiple myeloma antigens include CD56, TIM-3, CD33, CD123, and/or CD44. Antibodies or antigen-binding fragments directed against such antigens are known and include, for example, those described in U.S. Pat. Nos. 8,153,765; 8,603,477, 8,008,450; U.S. published application No. US20120189622; and published international PCT application Nos. WO2006099875, WO2009080829 or WO2012092612. In some embodiments, such antibodies or antigen-binding fragments thereof (e.g. scFv) can be used to generate a CAR.

In some embodiments, the antigen may be one that is expressed or upregulated on cancer or tumor cells, but that also may be expressed in an immune cell, such as a resting or activated T cell. For example, in some cases, expression of hTERT, survivin and other universal tumor antigens are reported to be present in lymphocytes, including activated T lymphocytes (see e.g., Weng et al. (1996) J Exp. Med., 183:2471-2479; Hathcock et al. (1998) J Immunol, 160:5702-5706; Liu et al. (1999) Proc. Natl Acad Sci., 96:5147-5152; Turksma et al. (2013) Journal of Translational Medicine, 11: 152).

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

In some embodiments as provided herein, an immune cell, such as a T cell, can be engineered to repress or disrupt the gene encoding the antigen in the immune cell so that the expressed antigen-specific receptor does not specifically bind the antigen in the context of its expression on the immune cell itself. Thus, in some aspects, this may avoid off-target effects, such as binding of the engineered immune cells to themselves, which may reduce the efficacy of the engineered in the immune cells, for example, in connection with adoptive cell therapy.

In some embodiments, such as in the case of an inhibitory CAR, the target is an off-target marker, such as an antigen not expressed on the diseased cell or cell to be targeted, but that is expressed on a normal or non-diseased cell which also expresses a disease-specific target being targeted by an activating or stimulatory receptor in the same engineered cell. Exemplary such antigens are MHC molecules, such as MHC class I molecules, for example, in connection with treating diseases or conditions in which such molecules become downregulated but remain expressed in non-targeted cells.

In some embodiments, the engineered immune cells can contain an antigen-specific receptor that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gpIOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens, such as gp120 (but see also Kuhlmann A S, Peterson C W, Kiem H P. Chimeric antigen receptor T-cell approaches to HIV cure. Curr Opin HIV AIDS. 2018 September; 13 (5): 446-453).

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the cells of the invention is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5 (215) (December 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

Example antigen-binding receptors include bispecific antibodies that are T-cell activating antibodies which bind not only the desired antigen but also an activating T-cell antigen such as CD3 epsilon.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive cell therapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-ITK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, the engineered immune cells can contain an antigen-specific receptor that targets one or more other antigens. In some embodiments, the one or more other antigens is a tumor antigen or cancer marker. Other antigen targeted by antigen-specific receptors on the provided immune cells can, in some embodiments, include orphan tyrosine kinase receptor ROR1, tEGFR, Her2, p95HER2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acetylcholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, NKG2D Ligands, NY-ESO-1, MART-1, gpIOO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the cells of the invention is genetically engineered to express two or more antigen-specific receptors on the cell, each recognizing a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5 (215) (December 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

In some contexts, the engineered cells include gene segments that cause the cells to be susceptible to negative selection in vivo, such as upon administration in adoptive cell therapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-ITK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In other embodiments of the invention, the cells, i.e., myeloid cells (typically dendritic cells or phagocytic cells such as macrophages), are not engineered to express recombinant antigen-specific receptors, but rather include naturally occurring antigen-specific receptors specific for desired antigens, such dendritic cells, monocytes, macrophages or their progenitors cultured in vitro or ex vivo, e.g., during the incubation step(s), to promote expansion of cells having particular antigen specificity.

### 3. Method for Obtaining Cells According to the Invention

### 3.1 Delivery of Nucleic Acids Encoding the Gene of Interest

### 3.1.1 Vectors and Methods for Cell Genetic Engineering

In some aspects, the genetic engineering involves introduction of a nucleic acid encoding the genetically engineered component or other component for introduction into the cell, such as a component encoding a nucleic acid encoding a gene of interest (e.g blc2) or a nucleic acid construct encoding a recognizing antigen receptor, in particular a CAR.

Generally, gene overexpression or CAR engineering into immune cells (e.g., dendritic cells, macrophages, monocytes, granulocytes, or their progenitors as defined above) requires that the cells be cultured to allow for transduction and expansion. The transduction may utilize a variety of methods, but stable gene transfer is required to enable sustained CAR or protein expression in clonally expanding and persisting engineered cells.

In some embodiments, gene transfer is accomplished by first stimulating cell growth, e.g., myeloid cell growth, proliferation, and/or activation, followed by transduction of the activated cells, and expansion in culture to numbers sufficient for clinical applications. In other embodiments, myeloid cells are transduced before differentiation and/or activation.

Various methods for the introduction of a gene of interest or of a genetically engineered components, e.g., antigen-specific receptors (such as CARs), are well known and may be used with the provided methods and compositions. Exemplary methods include those for transfer of nucleic acids encoding the receptors, including via viral, e.g., retroviral or lentiviral, transduction, transposons, and electroporation.

For example, a polynucleotide encoding a recombinant antigen recognizing receptor (typically a CAR) or a protein of interest (i.e., BCL2) can be cloned into a viral vector (e.g. a retroviral vector) and expression can be driven from a promoter to which the polynucleotide sequence of the gene of interest, or of the recognizing antigen receptor construct, can be operably linked. The promoter can be an endogenous promoter, a promoter specific for a target cell type of interest, or any other heterologous promoter of interest.

Non-viral vectors may be used as well.

The polynucleotide sequence can also be linked to appropriate control sequences allowing the regulation of its translation in a host cell.

The CAR can be constructed with an auxiliary molecule (e.g., a cytokine) in a single, multi cistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF−1 IRES, FGF-2 IRES, VEGF IRES, IGF-II IRES, NF-κβIRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g, 2A peptides, e.g., P2A, T2A, E2A and F2A peptides). In certain embodiments, any vector or CAR disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107: 77S-83S, 1995). Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997).

For initial genetic modification of an immunoresponsive cell to include a CAR, a retroviral vector is generally employed for transduction, however any other suitable viral vector or non-viral delivery system can be used. Typically, the vector is a lentiviral vector such as HIV-1-derived lentiviral vectors. Such embodiments are particularly efficient for transduction of dendritic cells and macrophages in particular derived from monocytes. Typically the target cells (e.g., monocyte derived macrophages or DCs) are co-transduced with helper particles derived from SIVmac that contain the viral protein Vpx (see Satoh T, Manel N. Gene transduction in human monocyte-derived dendritic cells using lentiviral vectors. Methods Mol Biol. 2013; 960:401-409 or Schroers R, Sinha I, Segall H, Schmidt-Wolf IG, Rooney C M, Brenner M K, Sutton R E, Chen S Y. Transduction of human PBMC-derived dendritic cells and macrophages by an HIV-1-based lentiviral vector system. Mol Ther. 2000 February;1 (2): 171-9).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are also known. Exemplary methods are described in, e.g., Wang et al. (2012) J. Immunother. 35 (9): 689-701; Cooper et al. (2003) Blood. 101:1637-1644; Verhoeyen et al. (2009) Methods Mol Biol. 506:97-114; and Cavalieri et al. (2003) Blood. 102 (2): 497-505.

In other embodiments, the vector can be an adenoviral derived from group B adenoviruses such as Ad35. This strategy may be relevant when macrophages are transduced.

Various promoters may be used to drive high expression of the nucleic acid sequence. The promoter may be a tissue-specific, ubiquitous, constitutive or inducible promoter. Preferred promoters are notably functional in the selected myeloid cell, for example a dendritic cell, a macrophage or a monocyte. In particular, preferred promoters are able to drive high expression the protein of interest (i.e., BCL2) or the chimeric construct (notably a CAR as previously defined) from viral vectors in myeloid cells, preferably human myeloid cells. For example, a promoter according to the present disclosure can be selected from phosphoglycerate kinase promoter (PGK), spleen focus-forming virus (SFFV) promoters, elongation factor-1 alpha (EF-1 alpha) promoter including the short form of said promoter (EFS), viral promoters such as cytomegalovirus (CMV) immediate early enhancer and promoter, retroviral 5′ and 3′ LTR promoters including hybrid LTR promoters, human ubiquitin promoter, MHC class I promoter, MHC class II promoter, and B2 microglobulin (B2m) promoter. The promoters are advantageously human promoters, i.e., promoters from human cells or human viruses such as spleen focus-forming virus (SFFV). Human ubiquitin promoter, MHC class I promoter, MHC class II promoter, and B2 microglobulin (B2m) promoter are more particular preferred. Preferably, the MHC class I promoter is an HLA-A2 promoter, an HLA-B7 promoter, an HLA-Cw5 promoter, an HLA-F, or an HLA-E promoter. In some embodiments the promoter is not a CMV promoter/enhancer, or is not a dectin-2 or MHCII promoter. Such promoters are well-known in the art and their sequences are available in sequence data base.

Non-viral approaches can also be employed for the expression of a protein in cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate (e.g., as described in Current Protocols in Molecular Biology, John Wiley & Sons, New York. N.Y.), DEAE dextran, transposition (see, e.g., Manuri et al. (2010) Hum Gene Ther 21 (4): 427-437; Sharma et al. (2013) Molec Ther Nucl Acids 2, e74; and Huang et al. (2009) Methods Mol Biol 506:115-126), electroporation (see, e.g., Chicaybam et al, (2013) PLOS ONE 8 (3): e60298 and Van Tedeloo et al. (2000) Gene Therapy 7 (16): 1431-1437), tungsten particle-facilitated microparticle bombardment (Johnston, Nature, 346:776-777 (1990)); strontium phosphate DNA co-precipitation (Brash et al., Mol. Cell Biol., 7:2031-2034 (1987)), and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers.

Alternatively, if a genomic clone is used as a therapeutic construct, regulation can be mediated by the cognate regulatory sequences or, if desired, by regulatory sequences derived from a heterologous source, including any of the promoters or regulatory elements described above. The resulting cells can then be grown under conditions similar to those for unmodified cells, whereby the modified cells can be expanded and used for a variety of purposes.

### 3.1.2 Genome Editing Methods

Any targeted genome editing methods can be used to place presently disclosed CARs at one or more endogenous gene loci of a presently disclosed immunoresponsive cell. In certain embodiments, a CRISPR system is used to deliver presently disclosed CARs to one or more endogenous gene loci of a presently disclosed immunoresponsive cell. In certain embodiments, zinc-finger nucleases are used to deliver presently disclosed CARs to one or more endogenous gene loci of a presently disclosed immunoresponsive cell. In certain embodiments, a TALEN system is used to deliver presently disclosed CARs to one or more endogenous gene loci of a presently disclosed immunoresponsive cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 3.2 Cell Preparation

Isolation of the cells includes one or more preparation and/or non-affinity-based cell separation steps according to well-known techniques in the field. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the cell preparation includes steps for freezing, e.g., cryopreserving, the cells, either before or after isolation, incubation, and/or engineering. Any of a variety of known freezing solutions and parameters in some aspects may be used.

The incubation steps can comprise culture, incubation, stimulation, activation, expansion and/or propagation.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a antigen-specific receptor.

The incubation conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of receptor present on a myeloid cell according to the present invention, such as in a non-limitative manner CD3 (in particular CD247, CD3z) and/or modified CD3 (notably modified CD247 or CD3z), the y subunit of Fc receptor (such as of FcERly), CD64, CD32, CD16, CD16a, CD16b, MEGF10, CD40, the Toll-like receptor/Interleukin (IL)-1 receptor (TLR/IL-1R) superfamily, members of the BAI family of phosphatidylserine receptor, such as BAI1, or members from the TAM family of phosphatidylserine receptors, such as MerTK. In some aspects, the agent turns on or initiates the intracellular signaling activation cascade in a myeloid cell, such as a monocyte, a macrophage, a dendritic cell, a progenitor thereof, or a progeny thereof (a subset population thereof). Such agents can include antibodies, such as those specific for a myeloid cell receptor component and/or costimulatory receptor, e.g., for example, bound to solid support such as a bead, and/or one or more cytokines (typically a cytokine cocktail).

In some aspects, incubation is carried out in accordance with techniques such as those described in US Patent No. 6,040, 1 77 to Riddell et al., Klebanoff et al., J Immunother. 2012; 35 (9): 651-660, Terakura et al., Blood. 2012; 1:72-82, and/or Wang et al. J Immunother. 2012,35 (9): 689-701.

In some embodiments, the myeloid cells, such as dendritic cells, macrophages, monocytes, granulocytes or one of their progenitors such as for example HSCs, MPs, CMPs, MDPs, CDPs, cMoPs, cDPs, or GMPs) are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human myeloid cells, such as dendritic cells, macrophages, monocytes, granulocytes or one of their progenitors such as for example HSCs, MPs, CMPs, MDPs, CDPs, cMoPs, cDPs, or GMPs), for example, at least about 25 degrees Celsius, generally at least about 30 degrees, and generally at or about 37 degrees Celsius.

In some aspects, the methods include assessing expression of one or more markers on the surface of the engineered cells or cells being engineered. In one embodiment, the methods include assessing surface expression of one or more target antigen (e.g., antigen recognized by the antigen-specific receptor) sought to be targeted by the adoptive cell therapy, for example, by affinity-based detection methods such as by flow cytometry. Thus in some embodiments, a population of myeloid cell progenitors for example HSCs, MPs, CMPs, MDPs, CDPs, cMoPs, cDPs, and/or GMPs are isolated, expanded and genetically modified to express a recombinant recognizing receptor as previously defined and/or to overexpress BCL2. Genetically modified progenitors are then activated and/or differentiated in a selected myeloid cell sub-population, such as dendritic cells, macrophages, monocytes, granulocytes or one of their sub-populations.

### 4. Composition of the Invention

The present invention also includes compositions containing the cells as described herein and/or produced by the provided methods. Typically, said compositions are pharmaceutical compositions and formulations for administration, preferably sterile compositions and formulations, such as for adoptive cell therapy.

A pharmaceutical composition of the invention generally comprises at least one engineered immune cell of the invention and a sterile pharmaceutically acceptable carrier.

As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can further be incorporated into the compositions. In some aspects, the choice of carrier in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001 to about 2% by weight of the total composition.

A pharmaceutical composition is formulated to be compatible with its intended route of administration.

The present invention also includes vaccine compositions containing the cells as described herein and/or produced by the provided methods and expressing or not the antigen recognizing receptor.

### 5. Therapeutic Methods

The present invention also relates to the cells as previously defined for their use in adoptive cell therapy (notably adoptive myeloid cell therapy), typically in the treatment of cancer in a subject in need thereof, but also in the treatment of infectious diseases and autoimmune, inflammatory or allergic diseases. Treatment of any of the diseases listed above under the “Antigen” section is contemplated.

Treatment”, or “treating” as used herein, is defined as the application or administration of cells as per the invention or of a composition comprising the cells to a patient in need thereof with the purpose to cure, heal, alleviate, relieve, alter, remedy, ameliorate, improve or affect the disease such as cancer, or any symptom of the disease (e.g., cancer). In particular, the terms “treat′ or treatment” refers to reducing or alleviating at least one adverse clinical symptom associated with the disease such as the cancer cancer, e.g., pain, swelling, low blood count etc.

With reference to cancer treatment, the term “treat′ or treatment” also refers to slowing or reversing the progression neoplastic uncontrolled cell multiplication, i.e. shrinking existing tumors and/or halting tumor growth. The term “treat′ or treatment” also refers to inducing apoptosis in cancer or tumor cells in the subject.

The immune cells, particularly myeloid cells (such as dendritic cells but also macrophages, monocytes or granulocytes) in which BCL2 has been overexpressed exhibit an increased survival in vivo. Furthermore, macrophages in which BCL2 has been overexpressed exhibit an increased cellular integrity. Thus, myeloid cells in which BCL2 has been overexpressed, which optionally have any of the other features described herein (e.g. expressing an antigen recognizing receptor and having typically targeted effector function directed against an antigen on a target cell that is specifically bound by the antigen recognizing receptor), may be administered at certain doses. For example, the myeloid cells has herein defined may be administered to adults at doses of less than about 10⁸ cells, less than about 5×10⁷ cells, less than about 10⁷ cells, less than about 5×10⁶ cells, less than about 10⁶ cells, less than about 5×10⁵ cells or less than about 10⁵ cells. The dose for pediatric patients may be about 100-fold less. In alternative embodiments, any of the immune cells (e.g. T-cells) described herein may be administered to patients at doses ranging from 10⁵ to 10⁹ cells, or 10⁵ to 10⁸ cells, or 10⁶ to 10⁸ cells.

The subject of the invention (i.e. patient) is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS). In some embodiments of the invention, said subject has a cancer, is at risk of having a cancer, or is in remission of a cancer.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Preferably, a cancer according to the invention is a cancer affecting the blood, bone marrow and lymphoid system as described above. Typically, the cancer is, or is associated with, multiple myeloma.

In some embodiments, the subject is suffering from or is at risk of an infectious disease or condition, notably of a chronic infectious disease, such as, but not limited to, viral, retroviral, bacterial, and protozoal infections, or immunodeficiency, associated with Cytomegalovirus (CMV), Epstein-Barr virus (EBV), adenovirus, BK polyomavirus, or human immunodeficiency virus (HIV).

In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

The present invention also relates to a method of treatment and notably an adoptive cell therapy, preferably an adoptive myeloid cell therapy, comprising the administration to a subject in need thereof of a modified cell as herein described or a population of cells (typically the myeloid cells are expanded in vitro after modification as herein disclosed) or a composition a previously described.

In some embodiments, the cells or compositions are administered to the subject, such as a subject having or at risk for a cancer or any one of the diseases as mentioned above. In some aspects, the methods thereby treat, e.g., ameliorate one or more symptom of, the disease or condition, such as with reference to cancer, by lessening tumor burden in a cancer expressing an antigen recognized by the engineered cell. Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338. In some embodiments, the cell therapy, e.g., adoptive cell therapy, e.g., adoptive myeloid cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive myeloid cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. In some embodiments, HLA matching is less important when the immune cell has been modified to reduce expression of endogenous TCR and HLA class I molecules.

Administration of at least one modified myeloid cell according to the invention to a subject in need thereof may be combined with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

With reference to cancer treatment, a combined cancer treatment can include but is not limited to cancer chemotherapeutic agents, cytotoxic agents, hormones, anti-angiogens, radiolabelled compounds, immunotherapy, surgery, cryotherapy, and/or radiotherapy or one or more additional adoptive cell therapy.

Conventional cancer chemotherapeutic agents include alkylating agents, antimetabolites, anthracyclines, topoisomerase inhibitors, microtubule inhibitors and B-raf enzyme inhibitors.

Alkylating agents include the nitrogen mustards (such as mechlorethamine, cyclophosphamide, ifosfamide, melphalan and chlorambucil), ethylenamine and methylenamine derivatives (such as altretamine, thiotepa), alkyl sulfonates (such as busulfan), nitrosoureas (such as carmustine, lomustine, estramustine), triazenes (such as dacarbazine, procarbazine, temozolomide), and platinum-containing antineoplastic agents (such as cisplatin, carboplatin, oxaliplatin).

Antimetabolites include 5-fluorouracil (5-FU), 6-mercaptopurine (6-MP), Capecitabine (Xeloda®), Cytarabine (Ara-C®), Floxuridine, Fludarabine, Gemcitabine (Gemzar®),

Hydroxyurea, Methotrexate, Pemetrexed (Alimta®).

Anthracyclines include Daunorubicin, Doxorubicin (Adriamycin®), Epirubicin. Idarubicin. Other anti-tumor antibiotics include Actinomycin-D, Bleomycin, Mitomycin-C, Mitoxantrone.

Topoisomerase inhibitors include Topotecan, Irinotecan (CPT-11), Etoposide (VP-16), Teniposide or Mitoxantrone. Microtubule inhibitors include Estramustine, Ixabepilone, the taxanes (such as Paclitaxel, Docetaxel and Cabazitaxel), and the vinca alkaloids (such as Vinblastine, Vincristine, Vinorelbine, Vindesine and Vinflunine). B-raf enzyme inhibitors include vemurafenib (Zelboraf), dabrafenib (Tafinlar), and encorafenib (Braftovi). Immunotherapy includes but is not limited to immune checkpoint modulators (i.e. inhibitors and/or agonists), cytokines, immunomodulating monoclonal antibodies, cancer vaccines.

Preferably, administration of cells in an adoptive myeloid cell therapy according to the invention is combined with administration of immune checkpoint modulators. Examples include inhibitors of (e.g. antibodies that bind specifically to and inhibit activity of) PD-1, CTLA4, LAG3, BTLA, OX2R, TIM-3, TIGIT, LAIR-1, PGE2 receptors, and/or EP2/4 Adenosine receptors including A2AR. Preferably, the immune checkpoint modulators comprise anti-PD-1 and/or anti-PDL-1 inhibitors (e.g., anti-PD-1 and/or anti-PDL-1 antibodies).

In some embodiments, the modified myeloid cell as herein described can be treated with a STING agonist (as described in PCT/EP2016/055738) before therapeutic use, in particular before adoptive transfer in a subject for therapeutic application. STING agonists notably include cyclic di-adenosine monophosphate (c-di-AMP), cyclic di-guanosine monophosphate (c-di-GMP), more specifically c [G (2′,5′) pG (3′,5′) p] and c [G (3′,5′) pG (3′,5′) p], and cyclic guanosine monophosphate-adenosine monophosphate (cGAMP), more specifically c [G (2′,5′) pA (3′,5′) p] and c [G (3′,5′) pA (3′,5′) p]. In alternative embodiments, the myeloid cell can transduced with a viral vector (such as lentiviral particles) wherein a STING agonist as above described in incorporated (see PCT/EP2016/055738). The present invention also relates to the use of a composition comprising the engineered immune cell as herein described for the manufacture of a medicament for treating a cancer, an infectious disease or condition, an autoimmune disease or condition, or an inflammatory disease or condition in a subject.

In some embodiment, myeloid adoptive cell therapy as herein defined may involve administration of one or more subpopulations of modified myeloid cells as herein defined (such as for example modified dendritic cell, modified macrophages and/or modified monocytes). One or more sub-populations of the same type of myeloid cell could also be combined. Said one or more populations of modified myeloid cells as herein defined my further be combined with administration of a “classical” T cell adoptive therapy (as described e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338). For example, modified dendritic cells, monocytes and/or macrophages as herein defined may be administered in combination with adoptive T cell therapy such as Chimeric Antigen Receptor (CAR) T cell therapy, Tumor-Infiltrating Lymphocyte (TIL) therapy, Engineered T Cell Receptor (TCR) therapy, and Natural Killer (NK) cell therapy. Further combination with other cancer treatment, such as immunotherapy (in particular immune checkpoint modulators) can also be envisioned.

In some embodiments, the modified myeloid cell as herein described, expressing or not a recombinant antigen receptor can be used in a vaccine. Typically, ex-vivo derived myeloid cells, typically DCs pulsed with antigenic viral or tumor peptides, such as tumor-associated antigens (TAAs) (or tumor cell lysates) can be reinfused ex vivo in a patient in need thereof. In some embodiments, the modified myeloid cells, typically the modified DCs can be further stimulated with a defined maturation cocktail.

### EXAMPLES

### Materials and Methods

### Cell Lines

HEK 293 FT cells were obtained from Thermo Fisher Scientific. Cell line validation was performed by STR and POWERPLEX 16HS analysis for both cell lines. All cell lines were routinely screened for the presence of mycoplasma (Eurofins).

### Plasmids and Constructs

CMV-VSVG, pSIV3+ and psPAX2 were described (Manel et al., Nature, 2010, 467, 214-217). pTRIP-SFFV-mtagBFP-2A was described (Cerboni et al., J. Exp. Med., 2017, 214, 1769-1785). pTRIP-SFF-tagRFP657-2A was described (Bhargava et al., Cell Reports, 2021, 36, 109763-). pCDH1 was described (Gea-Mallorqui et al., Front. Microbiol., 2020, 11, doi: 10.3389/fmicb 2020.01603). Red firefly luciferase gene was cloned into pTRIP-BFP-2a vector from pMCS-Red Firefly Luc (Thermo Scientific, ref: 16155) to produce pTRIP-BFP-2a-RedLuc-pEF1-YP.

BCL2 was cloned from HDV-BCL2-IRES-dsRED (S. A. Rawlings, F. Alonzo, 3rd, L. Kozhaya, V. J. Torres, D. Unutmaz, Elimination of HIV-1-Infected Primary T Cell Reservoirs in an In Vitro Model of Latency. PloS one 10,e0126917 (2015) 10.1371/journal.pone.0126917) into pTRIP-SFFV-mtagBFP-2A, resulting in pTRIP-SFFV-mtagBFP-2A-BCL2 and into pTRIP-SFF-tagRFP657-2A resulting in pTRIP-SFFV-tagRFP657-2A-BCL2.

CAR constructs that expressed CD19BBz codon optimized, CD19z and CD19trunc (truncated form) were cloned from synthetic DNA sequence into pTRIP-SFFV-mtagBFP-2A-BCL2; resulting in pTRIP—SFFV-CD19BBzCo-2A-tagBFP-2A-BCL2, pTRIP—SFFV-CD19z-2A-tagBFP-2A-BCL2 and pTRIP—SFFV-CD19trunc-2A-tagBFP-2A-BCL2. Synthetic DNA sequence came from the sequence of CD19 constructed already described (Brogdon et al, US2014271635A1). CD19z was also cloned in the lentivector pCDH1, resulting in pCDH1-CAR-CD3 (SEQ ID NO: 9).

In final constructs, the entire DNA fragments originating from PCR or synthesis and used for cloning were fully verified by sequencing encompassing the restriction sites. Plasmid DNA was purified using with the low endotoxin HiPure plasmid kit (Thermo Fisher Scientific).

### Cell Line Cultivation and Isolation of Monocytes

Human peripheral blood mononuclear cells (hPBMCs) were isolated from blood obtained from Leukocyte Reduction System Chamber (LRSC) including the tubing from healthy human donors (approved by the Institut National de la Santé et de la Recherche Médicale ethics committee) using Ficoll-Paque PLUS (GE) in SepMate™ tubes and taken up in RPMI medium supplemented with 10% FBS (Corning), Penicillin-Streptomycin, Gentamicin (50 μg/ml, Gibco), HEPES (Gibco) (DC medium) and stored at 4° C. until further processing. CD14+ cells were isolated by positive selection with anti-human CD14 magnetic beads (Miltenyi) in LS columns on the manual QuadroMACS™ separator from PBMCs. Purity was checked by flow cytometry using an anti-CD14 (eBioscience), anti-DC-SIGN (R&D Systems) antibody and purity was superior to 97%. To obtain monocyte-derived dendritic cells (MDDCs), CD14+ cells were cultured in DC medium supplemented with human GM-CSF (Miltenyi) at 10 ng/ml, and IL-4 (Miltenyi) at 50 ng/ml for 5 days. Absence of mycoplasma contamination during the course of the study was confirmed regularly (Eurofins).

### Lentiviral Vector Production

Lentiviral vectors were produced by transfection of 0.8 million HEK 293 FT cells (passage 3-22) per 6-well with 3 μg DNA and 8 μl TransIT-293 (Mirus Bio) per well; for VSV-G pseudotyped SIVmac VLPs (SIVVLPs), 0.4 μg CMV-VSVG and 2.6 μg pSIV3+ were transfected; for VSV-G pseudotyped pTRIP or pCDH1 viruses (for overexpression), 0.4 μg CMV-VSVG, 1 μg psPAX2 and 1.6 μg of lentivector of interest were combined. The morning after transfection medium was replaced with fresh DC medium. Viral supernatants were harvested after 24 hours, filtered at 0.45 μm, and used directly for transduction of monocytes. Of note, lentivirus-containing supernatants did not alter dendritic cell differentiation or lead to their activation in the absence infection/stimulation.

### Transduction of Monocytes

For in vitro experiment and MVA infection, after CD14⁺ isolation, monocytes were seeded in 6-well plates at 3 million per well in 1 mL in DC medium supplemented with human GM-CSF (Miltenyi) at 70 ng/ml, IL-4 (Miltenyi) at 350 ng/ml and protamine (7 μg/ml). Viral supernatants were added to each well: 3 mL SIVmac VLPs, 3 mL pTRIP-SFFV-mtagBFP-2A or pTRIP-SFFV-mtagBFP-2A-BCL2. Transduced cells were incubated at 37° C. for 4 to 5 days.

For in vivo BCL2 experiment, after CD14+ isolation, monocytes were seeded in 6-well plates at 3 million per well in 1 mL in DC medium supplemented with human M-CSF (Miltenyi) at 1 mg/ml, IL-4 (Miltenyi) at 50 ng/ml, TNFα (RnDsystems) at 50 ng/ml and protamine (10 μg/ml). Viral supernatants were added to each well: 3 mL SIVmac VLPs, 3 mL pTRIP-BFP-2A-RedLuc-pEF1-YP and 3 mL pTRIP-SFFV-tagRFP657-2A or pTRIP-SFFV-tagRFP657-2A-BCL2. Transduced cells were incubated at 37° C. for 4 to 5 days.

For in vivo CAR-BCL2 experiment, after CD14+ isolation, monocytes were seeded in 6-well plates at 3 million per well in 1 mL in DC medium supplemented with human M-CSF (Miltenyi) at 1 mg/ml, IL-4 (Miltenyi) at 50 ng/ml, TNFα (RnDsystems) at 50 ng/ml and protamine (10 μg/ml). Viral supernatants were added to each well: 3 mL SIVmac VLPs, 3 mL pTRIP-BFP-2A-RedLuc-pEF1-YP and 3 mL pTRIP-SFFV-tagBFP-2A or pTRIP—SFFV-CD19BBzCo-2A-tagBFP-2A-BCL2 or pTRIP—SFFV-CD19z-2A-tagBFP-2A-BCL2 or pTRIP—SFFV-CD19trunc-2A-tagBFP-2A-BCL2. Transduced cells were incubated at 37° C. for 4 to 5 days.

For in vitro BCL2 experiment in MDDCs, after CD14+ isolation, monocytes were seeded in 6-well plates at 3 million per well in 1 mL in DC medium supplemented with human GM-CSF (Miltenyi) at 70 ng/ml, IL-4 (Miltenyi) at 350 ng/ml and protamine (7 μg/ml). Viral supernatants were added to each well: 3 mL SIVmac VLPs, 3 mL pTRIP-SFFV-mtagBFP-2A or pTRIP-SFFV-mtagBFP-2A-BCL2. Two wells (so 6 million cells) were performed for each condition. Transduced cells were incubated at 37° C. At day 4 and 10 of MDDCs differentiation, cells were harvested, counted and resuspended in fresh DC medium supplemented with GM-CSF (20 ng/ml) and IL-4 (100 ng/ml) at a concentration of 1 million per ml and plated on one well in 6-well plates (or in 12-well plate when less of 2 million cells). At day 7, 17, 24 and 32, cells were harvested, counted and put back in culture.

For in vitro BCL2 experiment in MDMs, after CD14+ isolation, monocytes were seeded in 10 cm cell culture dishes at 7 million per dish in 1 mL in with human M-CSF (Miltenyi) at 750 ng/ml and protamine (15 μg/ml). Viral supernatants were added to each well: 7 mL SIVmac VLPs, 7 mL pTRIP-SFFV-mtagBFP-2A or pTRIP-SFFV-mtagBFP-2A-BCL2. Transduced cells were incubated at 37° C. At each days analyzed, cells were detached by Accutase (Sigma), harvested, counted and resuspended in fresh medium supplemented with M-CSF (50 ng/ml) at a concentration of 1 million per ml and plated on one well in 6-well plates (maximum 2 millions cells per P6 wells or in 12-well plate when less of 1 million cells).

For in vitro CAR-BCL2 experiment in MDMs, after CD14+ isolation, monocytes were seeded in 10 cm cell culture dishes at 7 million per dish in 1 mL in with human M-CSF (Miltenyi) at 750 ng/ml and protamine (15 μg/ml). Viral supernatants were added to each well: 7 mL SIVmac VLPs, 7 mL pTRIP-SFFV-mtagBFP-2A or pTRIP-SFFV-mtagBFP-2A-BCL2 and 7 mL of pCDH1-CAR-CD3.

To quantify live cell numbers and to measure Cell Integrity (%), the AO/PI Cell Viability Kit (Labtech-Acridine Orange/Propidium lodide stain) were used.

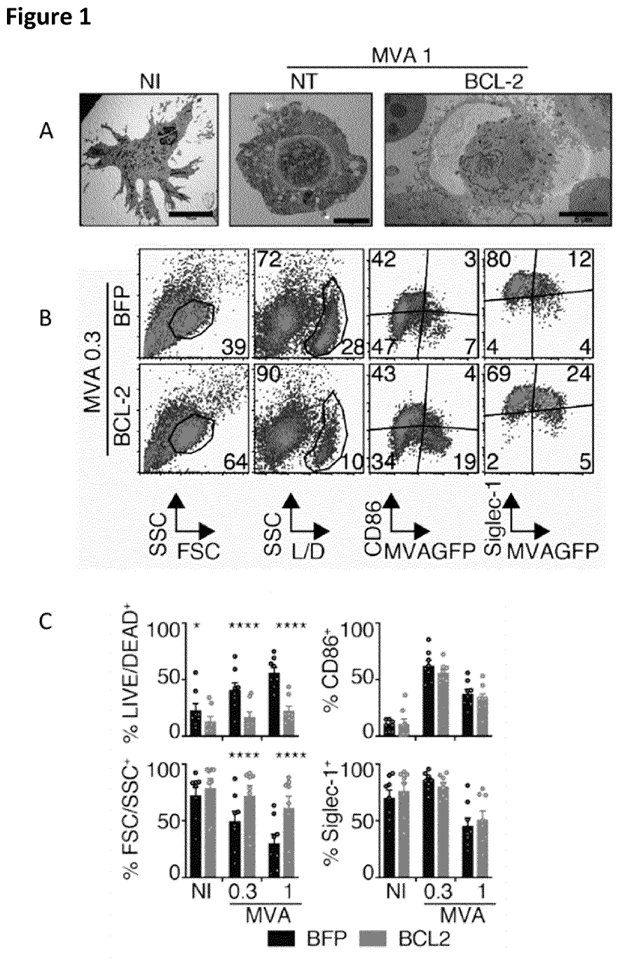

### MVA Infection (FIG. 1)

At day 5 of MDDC differentiation, cells were harvested, counted and resuspended in fresh DC medium supplemented with GM-CSF (20 ng/ml) and IL-4 (100 ng/ml) at a concentration of 1 million per ml. 100 μl of MDDCs were seeded in round-bottom 96-well plates and stimulated/infected with 100 μl of MVA dilutions. MVA infection was performed with increasing MOls at increments of ⅓ ranging from 0.01-1. After 24 hours of infection, cell-surface immunolabeling with an anti-CD86 (eBioscience) and an anti-SIGLEC1 (Miltenyi Biotec) was performed together with a fixable viability dye (eBiosience).

### Electron Microscopy (FIG. 1)

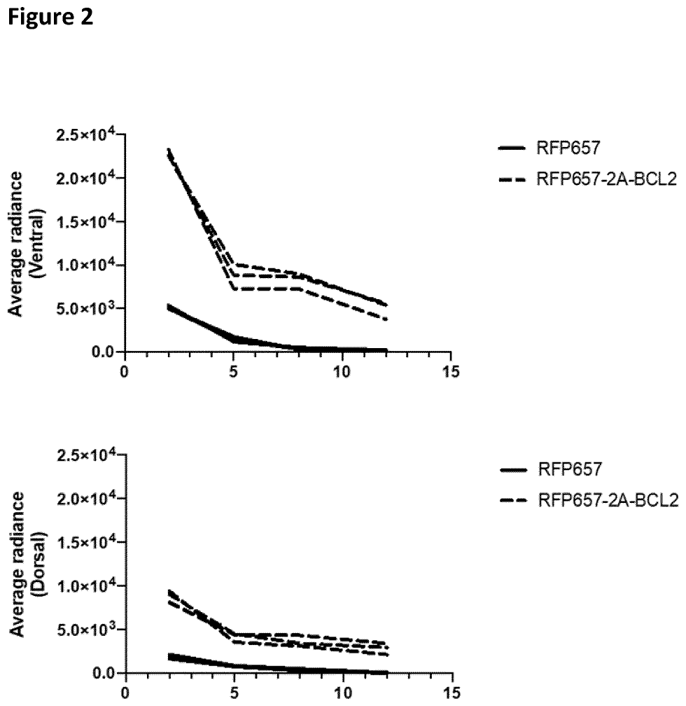

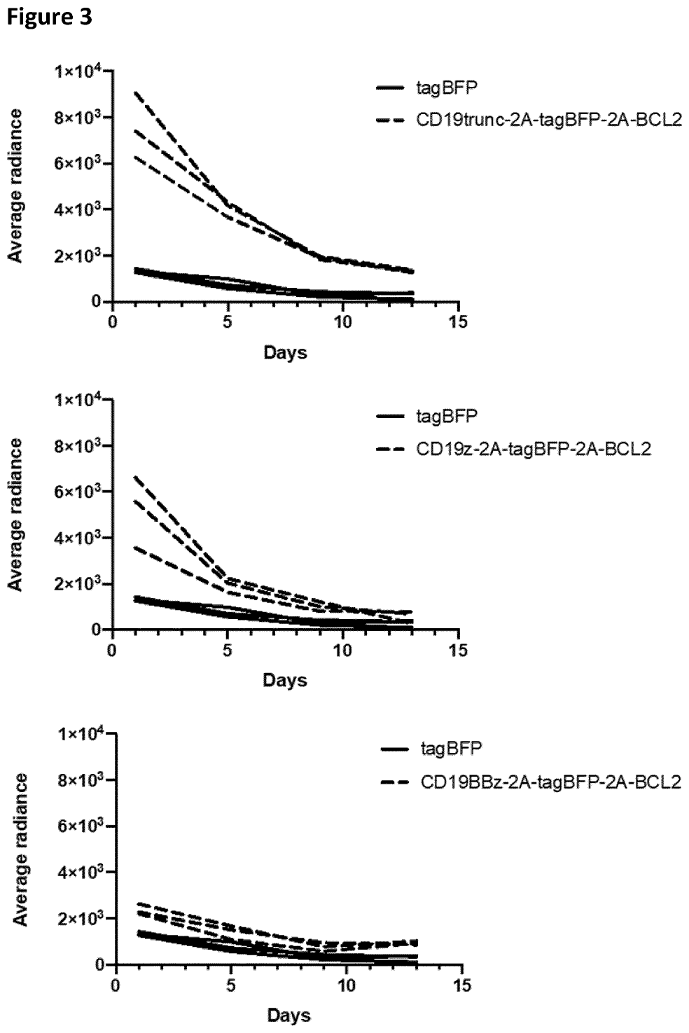

For electron microscopy MDDCs were infected for 24 hours in 96 well round-bottom plates and then transferred onto poly-L-lysin treated coverslips. Upon attachment for 30 min at 37° C., cells were fixed in 1 ml of fixative (2.5% glutaraldehyde in 0.1 M cacoldylate buffer, pH 7.4) for 1 hour, post fixed for 1 hour with 2% buffered osmium tetroxide, dehydrated in a graded series of ethanol solution, and then embedded in epoxy resin. Images were acquired with a digital camera Quemesa (SIS) mounted on a Tecnai Spirit transmission electron microscope (FEI Company) operated at 80 kV. Myeloid cells overexpressing BCL2 for in vivo infection (FIGS. 2 and 3)

As described above, at day 4 or 5 after monocytes transduction and differentiation, cells were harvested, counted and resuspended in fresh DC medium supplemented with M-CSF (100 ng/ml), IL-4 (5 ng/ml) TNFα (RnDsystems) at 5 ng/mL at a concentration of 1 million per ml. Levels of expression of the transduced vectors on cells were analyzed by measuring RFP657 and BFP expression. Differentiation and activation were analyzed by cell-surface staining using anti-CD14 (eBioscience), anti-DC-SIGN (R&D Systems), anti-CD1a (Miltenyi Biotec) and anti-CD16 (BD for differentiation, and anti-CD86 (eBioscience) anti-SIGLEC1 (Miltenyi Biotec) for activation. Cells were analyzed using a flow cytometer.

### Mice and Adoptive Transfer Experiment

The day of the adoptive transfer, myeloid cells were harvested, washed and resuspended in PBS. For in vivo BCL2 experiment, NSG females were injected intravenously with 6.5×10⁶ cells in 100 μL of PBS via the lateral tail vein (n=3). For in vivo CAR-BCL2 experiment, NSG females were injected intravenously with 4.5×10⁶ cells in 100 μL of PBS via the lateral tail vein (n=3). To track myeloid cells, bioluminescence imaging was performed at different timepoints (IVIS, PerkinElmer). Images were analyzed using Live Image software (PerkinElmer).

### Results

FIG. 1—the Results Show that Overexpression of BCL2 Specifically Inhibit Apoptosis in MDCCs In Vitro.

Indeed, BCL2 expressing MDDCs were completely protected against MVA associated apoptosis.

Electron micrographs revealed that MDDCs exhibited a variety of morphological characteristics of apoptosis 24 hpi (hours post infection) with MVA, i.e. rounding up, retraction of pseudopods, reduction of cellular volume, chromatin condensation, plasma membrane blebbing, cytoplasm fragmentation with formation of apoptotic bodies, (compare left image with middle image of FIG. 1A). BCL2 expressing MDDCs did not generally exhibit these features (FIG. 1A, right image).

The results further showed that BCL2 expressing MDDCs had enhanced MVAGFP expression, but no change in CD86 and Siglec-1 induction (FIG. 1B-C).

### FIG. 2—BCL2 Overexpression Increases In Vivo Persistence

Two days after the adoptive transfer of myeloid cells, 5-fold increased bioluminescence signal was detected in mice that received BCL2-overexpressing cells compared to control cells, from both ventral and dorsal views (FIG. 2). IVIS imaging suggested that luciferase expressing myeloid cells were homed to the liver, lung and bone marrow. At day 12, bioluminescence was only detected in the BCL2-overexpressing group. This was confirmed by flow cytometry ex vivo at day 13, when mice were sacrificed, clearly showing their increased survival capacities in NSG mice. The results show for the first time that myeloid cell overexpressing BLC2 show increased survival after adoptive transfer therefore representing an important improvement in adoptive cell therapy.

### FIG. 3—CAR-BCL2 Overexpression Increases In Vivo Persistence

Inventors next asked if BCL2 would increase survival in vivo in the context of CAR expression on myeloid cells. Three CARs specific for human CD19 were tested, one containing in the intracellular domain the portion of CD3z with its three ITAMs (CD19z), one containing the endodomain of 41BB in addition to the CD3z portion (CD19BBz) and one truncated CAR lacking an intracellular domain (CD19trunc). After in vivo injection, BCL2 expression conferred inscrease persistence for all CAR tested (FIG. 3). This shows for the first time that CAR-positive human myeloid cells overexpressing BCL2 survive better than control cells in a complex physiologic environment.

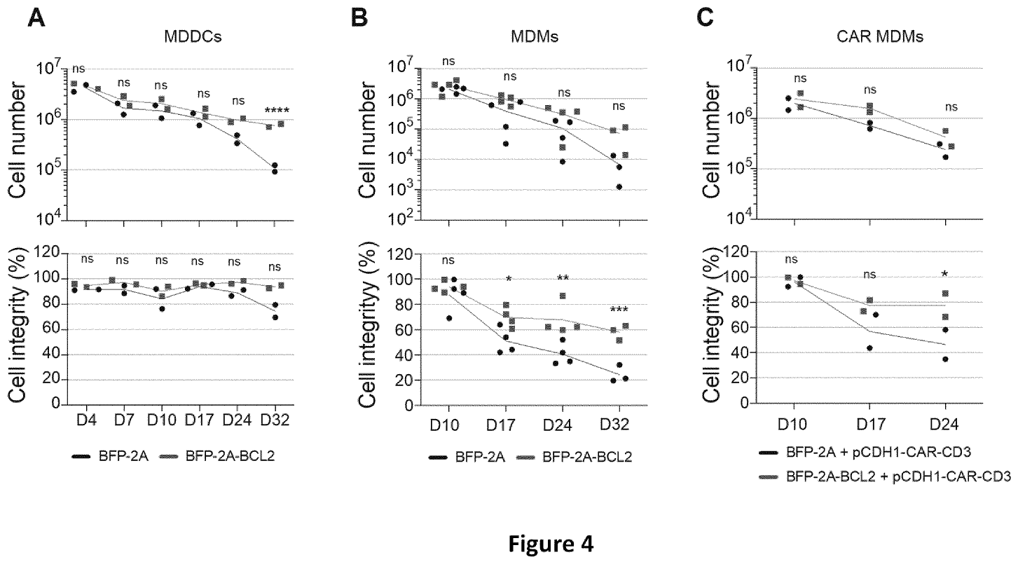

### FIG. 4—BCL2 Overexpression Unexpectedly Increases Cellular Integrity, but not Survival, in MDMs or CAR-MDMs.

The inventors examined if BCL2 would increase survival (resistance from spontaneous apoptosis) in macrophages (MDMs) as compared to previously tested dendritic cells (MDDCs). Cell number was measured over time to detect the effect of BCL2 on survival. Cellular integrity (permeability of the plasma membrane) was also measured using a cell permeability dye. In MDDCs, BCL2 increased the number of surviving cells at day 32 of long-term cultures (FIG. 4A, top), indicating resistance to spontaneous apoptosis, as previously observed. However, the integrity of the cells, as measured by a cell permeability dye was unaffected by BCL2 (FIG. 4A, bottom). In MDMs, in contrast, BCL2 did not increase the number of surviving cells after long term culture (FIG. 4B, top, day 10 to day 32). Instead, unexpectedly, BCL2 significantly limited the staining of cells by a cell permeability dye (FIG. 4B, bottom, day 17 to day 32). This indicates that BCL2, in MDMs, enhances the integrity of cells, independently from cell survival and protection against apoptosis. Next, it was tested if this also applied to CAR-expressing macrophages (CAR-MDMs). Indeed, similarly, BCL2 did not enhance the survival of CAR-MDMs (FIG. 4C, top). However, using the cell permeability marker, it was found that BCL2 enhanced integrity of the CAR-MDMs (FIG. 4C, bottom). In conclusion, BCL2 has an unexpected effect in MDMs and CAR-MDMs (CAR-Macrophages): it does not significantly protect from cell death (no increase in cell number), but it enhances the integrity of cells (cells are less permeable to a staining dye).

TABLE 1
Sequence listing

| SEQ ID | sequence |
|---|---|
| 1 | BLC2 protein, UniProtKB - P10415 (BCL2_HUMAN), |
| | canonical isoform alpha |
| 2 | BLC2 protein, UniProtKB - P10415 (BCL2_HUMAN)), |
| | isoform beta |
| 3 | CAR CD19BBzCo |
| 4 | CAR CD19 trunc |
| 5 | CAR CD19z |
| 6 | pTRIP-SFFV-CD19BBzCo-2A-tagBFP-2A-BCL2 |
| 7 | pTRIP-SFFV-CD19trunc-2A-tagBFP-2A-BCL2 |
| 8 | pTRIP-SFFV-CD19z-2A-tagBFP-2A-BCL2 |
| 9 | pCDH1-CAR-CD3 |

1-16. (canceled)

17. An isolated modified myeloid cell, a progenitor thereof, or a progeny thereof, encoding an antigen recognizing receptor, wherein said myeloid cell or progenitor thereof has been further modified to overexpress BCL2.

18. The modified myeloid cell or a progenitor thereof of claim 17, wherein the progenitor is a myeloid progenitor, a granulocyte monocyte dendritic cell progenitor (GMDP), a monocyte dendritic cell progenitor (MDP) or a common dendritic cell progenitor (cDP).

19. The modified myeloid cell of claim 17, wherein the myeloid cell is a macrophage.

20. The modified myeloid cell of claim 17, wherein the myeloid cell is a granulocyte, a monocyte, a macrophage or a dendritic cell having targeted effector activity.

21. The modified myeloid cell of claim 20, wherein the targeted effector activity is directed against an antigen on a target cell that is specifically bound by the antigen recognizing receptor.

22. The modified myeloid cell of claim 20, wherein the targeted effector activity is selected from the group consisting of phagocytosis, targeted cellular cytotoxicity, antigen presentation, cytokine secretion, and activation of cell migration.

23. The modified myeloid cell or progenitor thereof of claim 17, wherein the antigen recognizing receptor is recombinantly expressed.

24. The modified myeloid cell or progenitor thereof of claim 23, wherein the recombinantly expressed antigen recognizing receptor is a chimeric antigen receptor (CAR).

25. The modified myeloid cell or progenitor thereof of claim 23, wherein the antigen recognizing receptor is expressed from a vector.

26. The modified myeloid cell or progenitor thereof of claim 23, wherein the antigen recognizing receptor is expressed from a lentivirus vector.

27. The modified myeloid cell or progenitor thereof of claim 17, wherein the expression of SIRPa is disrupted or downregulated.

28. The modified myeloid cell or progenitor thereof of claim 17, wherein the antigen is a tumor antigen selected from the group consisting of CD19, MUC16, MUC1, CA1X, CEA, CD8, CD7, CD 10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD3, ITER-2, hTERT, IL-13R-a2, K-light chain, KDR, LeY, LI cell adhesion molecule, MAGE-A1, Mesothelin, ERBB2, MAGEA3, p53, MARTI, GPI00, Proteinase3 (PR1), Tyrosinase, Survivin, hTERT, EphA2, NKG2D ligands, NY-ESO-1, oncofetal antigen (hST4), PSCA, PSMA, ROR1, TAG-72, VEGF-R2, WT-I, BCMA, CD123, CD44V6, NKCS1, EGF1R, EGFR-VIII, and CD99, CD70, ADGRE2, CCR1, LILRB2, PRAME, CCR4, CD5, CD3, TRBC1, TRBC2, TIM-3, Integrin B7, ICAM-I, CD70, Tim3, CLEC12A and ER.

29. The modified myeloid cell or progenitor thereof of claim 17, wherein the antigen recognizing receptor is a CAR comprising a signalling domain selected from CD3, the y subunit of Fc receptor (such as of FcaRly), CD64, CD32, CD32b, CD32c, CD16, CD16a, CD16b, MEGF10, CD40, the Toll-like receptor/Interleukin (IL)-1 receptor (TLR/IL-1R) superfamily, members of the BAI family of phosphatidylserine receptor, and members from the TAM family of phosphatidylserine receptors.

30. The modified myeloid cell or progenitor thereof of claim 17, wherein the antigen recognizing receptor is a CAR comprising a TLR signalling domain chosen from the Toll/interleukin receptor homology domain and any intracellular domain interacting with the MyDDosome and/or theTRIFosome clusters.

31. A pharmaceutical composition comprising at least a modified myeloid cell or a progenitor thereof according to claim 17 and a pharmacological excipient.

32. A method for producing a modified myeloid cell or a progenitor thereof, the method comprising a step consisting in generating a modified myeloid cell or a progenitor thereof overexpressing BCL2 compared to a non-modified myeloid cell or progenitor thereof; and further comprising a step consisting in recombinantly expressing in said cell an antigen recognizing receptor.

33. A method for treating a subject in need thereof, comprising administering a modified myeloid cell or a progenitor thereof according to claim 17 to the subject.

34. The method of claim 33, wherein the subject is suffering from a disease chosen from a cancer, an auto-immune disease or an infectious disease.

35. The method of claim 33, which is adoptive cellular therapy.

36. The modified myeloid cell or a progenitor thereof of claim 17, wherein said cell or progenitor thereof is autologous.


SEQUENCE LISTING (9 sequences)

SEQ ID NO: 1 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 2 - protein, 205 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 3 - DNA, 1458 bp, Artificial Sequence
  synthetic polynucleotide (CAR CD19BBzCo)
atggcccttcctgttactgctcttctgcttcctctcgctctgcttctgcatgctgctagacctgatatacaaatgactcaaacgacgagctctcttagcgcatcccttggtgatcgagtgactataagctgtcgtgcttcccaagatatctctaagtacttgaactggtaccaacaaaagcctgacggcacagtcaagctgcttatttatcacactagcaggctgcatagcggcgtgcccagccgttttagcggatcaggatccgggaccgactacagccttactatctccaatcttgaacaggaagacatcgctacatatttctgtcagcaaggcaacactctcccttatacttttggcggcggcacaaaacttgaaattacgggtggtggcggatcaggtggggggggctcaggggggggtggctccgaagtcaagttgcaagaaagtgggcccgggcttgtagcccctagtcaatctctctctgtgacttgtaccgtgtctggagtgagcctgccggattacggagtgagttggatacggcaaccaccccggaaaggactcgaatggttgggcgtgatctggggctctgagactacttattacaactccgccttgaagtcaaggctcacgataattaaagataatagcaaatcccaggtgtttctgaagatgaatagccttcagacggacgatacggcaatctattattgcgcaaagcactactattatggcggctcctacgctatggattattggggacaggggacatctgtgacagtgtctagtacgactacccccgctccacgcccgcctactcctgcacctactatagcctcacaaccgctctctctcaggcctgaagcatgtcgtcctgcagccggtggtgctgtccatacaagaggcttggattttgcttgcgacatatatatttgggctccgctcgctggcacctgcggagtgctcttgttgtccctcgtcattacactgtattgtaagcgcgggcggaagaagttgctctacattttcaagcagcctttcatgcggcctgtgcagaccacccaggaagaggacgggtgctcttgtcggttccctgaggaagaggagggagggtgcgagctccgggtcaaattttcacgcagtgccgatgctccagcttataaacaaggacaaaatcaactgtataatgaacttaacttgggccgcagggaagaatatgatgtgctcgataaacgcagagggagagatccggaaatgggtggcaaacccaggcggaagaatccacaagagggattgtataacgagcttcaaaaggacaaaatggcagaagcatatagcgaaatcggaatgaagggagaaagacgacgaggaaaaggacatgacgggctgtatcaaggcctgagcaccgctacaaaagatacttatgatgctctgcatatgcaagcattgccaccaaga

SEQ ID NO: 4 - DNA, 996 bp, Artificial Sequence
  synthetic polynucleotide (CAR CD19trunc)
atggctctgcccgttactgcactccttctcccactcgctcttctcctgcatgctgctcggcctgatattcaaatgactcaaaccactagtagcctttccgcttcactcggcgatcgggtgacaatttcctgtcgggcctcacaagatatctccaagtaccttaactggtaccaacaaaagcccgacggcaccgtaaagctgttgatatatcacaccagtcgcttgcatagcggcgtgccgagccggttttcaggatccggcagtgggactgactacagtttgacgatatccaatttggaacaggaagacatagctacgtatttctgtcagcaaggcaacactctgccttatacctttggcggcggaactaaactcgaaattactggaggaggcggttccggtggaggaggcagcggggggggtggctcagaagtcaagttgcaagaaagcggcccaggactcgttgccccgtcacaatccctctctgtgacttgcacagtgtctggcgtatctcttccagattacggggtgagttggataagacaaccacctcggaaaggactcgaatggctcggcgtcatctggggatccgagactacgtattacaactctgcactgaagagccggctcacaattattaaagataatagtaaatcccaggtgtttctgaagatgaatagcctgcagacggacgataccgcaatctattattgcgcaaagcactactattatggcgggtcatacgccatggattattggggtcagggtactagcgttacggtgagtagcacaactaccccggcacccaggccgcctacgccagccccaactattgcttctcaacctctttctcttagacctgaagcctgtagacctgcagccggcggtgcggtccatacccggggactcgattttgcttgcgacatttatatttgggctcctctggctggaacctgcggcgtgctgctgctcagtcttgtcattactctgtattgt

SEQ ID NO: 5 - DNA, 2097 bp, Artificial Sequence
  synthetic polynucleotide (CAR CD19z)
atggctctgcccgttactgcactccttctcccactcgctcttctcctgcatgctgctcggcctgatattcaaatgactcaaaccactagtagcctttccgcttcactcggcgatcgggtgacaatttcctgtcgggcctcacaagatatctccaagtaccttaactggtaccaacaaaagcccgacggcaccgtaaagctgttgatatatcacaccagtcgcttgcatagcggcgtgccgagccggttttcaggatccggcagtgggactgactacagtttgacgatatccaatttggaacaggaagacatagctacgtatttctgtcagcaaggcaacactctgccttatacctttggcggcggaactaaactcgaaattactggaggaggcggttccggtggaggaggcagcggggggggtggctcagaagtcaagttgcaagaaagcggcccaggactcgttgccccgtcacaatccctctctgtgacttgcacagtgtctggcgtatctcttccagattacggggtgagttggataagacaaccacctcggaaaggactcgaatggctcggcgtcatctggggatccgagactacgtattacaactctgcactgaagagccggctcacaattattaaagataatagtaaatcccaggtgtttctgaagatgaatagcctgcagacggacgataccgcaatctattattgcgcaaagcactactattatggcgggtcatacgccatggattattggggtcagggtactagcgttacggtgagtagcacaactaccccggcacccaggccgcctacgccagccccaactattgcttctcaacctctttctcttagacctgaagcctgtagacctgcagccggcggtgcggtccatacccggggactcgattttgcttgcgacatttatatttgggctcctctggctggaacctgcggcgtgctgctgctcagtcttgtcattactctgtattgtcgcgttaaatttagtcggtcagctgatgcacctgcttataaacaagggcaaaatcaactctacaatgaactgaacctcggccgccgcgaagaatatgacgtactcgataaacgcaggggtcgagatcccgaaatgggcgggaaaccccgacggaagaatccccaagagggtctctataacgagcttcaaaaggacaaaatggccgaagcatatagcgaaataggaatgaagggagaaaggcgtcgcggaaaaggtcatgacggtctgtatcaaggcttgtctaccgctacaaaagatacgtatgatgctttgcatatgcaagctctcccaccaagaggtagtggtgccacaaacttttcactcctcaaacaagccggtgatgttgaagaaaatccgggtccaatgtctgaactcatcaaagaaaatatgcatatgaaactgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 6 - DNA, 12659 bp, Artificial Sequence
  synthetic polynucleotide (pTRIP-SFFV-CD19BBzCo- 2A-tagBFP-2A-BCL2)
acataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcgggctagcgccaccatggcccttcctgttactgctcttctgcttcctctcgctctgcttctgcatgctgctagacctgatatacaaatgactcaaacgacgagctctcttagcgcatcccttggtgatcgagtgactataagctgtcgtgcttcccaagatatctctaagtacttgaactggtaccaacaaaagcctgacggcacagtcaagctgcttatttatcacactagcaggctgcatagcggcgtgcccagccgttttagcggatcaggatccgggaccgactacagccttactatctccaatcttgaacaggaagacatcgctacatatttctgtcagcaaggcaacactctcccttatacttttggcggcggcacaaaacttgaaattacgggtggtggcggatcaggtggggggggctcaggggggggtggctccgaagtcaagttgcaagaaagtgggcccgggcttgtagcccctagtcaatctctctctgtgacttgtaccgtgtctggagtgagcctgccggattacggagtgagttggatacggcaaccaccccggaaaggactcgaatggttgggcgtgatctggggctctgagactacttattacaactccgccttgaagtcaaggctcacgataattaaagataatagcaaatcccaggtgtttctgaagatgaatagccttcagacggacgatacggcaatctattattgcgcaaagcactactattatggcggctcctacgctatggattattggggacaggggacatctgtgacagtgtctagtacgactacccccgctccacgcccgcctactcctgcacctactatagcctcacaaccgctctctctcaggcctgaagcatgtcgtcctgcagccggtggtgctgtccatacaagaggcttggattttgcttgcgacatatatatttgggctccgctcgctggcacctgcggagtgctcttgttgtccctcgtcattacactgtattgtaagcgcgggcggaagaagttgctctacattttcaagcagcctttcatgcggcctgtgcagaccacccaggaagaggacgggtgctcttgtcggttccctgaggaagaggagggagggtgcgagctccgggtcaaattttcacgcagtgccgatgctccagcttataaacaaggacaaaatcaactgtataatgaacttaacttgggccgcagggaagaatatgatgtgctcgataaacgcagagggagagatccggaaatgggtggcaaacccaggcggaagaatccacaagagggattgtataacgagcttcaaaaggacaaaatggcagaagcatatagcgaaatcggaatgaagggagaaagacgacgaggaaaaggacatgacgggctgtatcaaggcctgagcaccgctacaaaagatacttatgatgctctgcatatgcaagcattgccaccaagaggtagtggtgccacaaacttttcactcctcaaacaagccggtgatgttgaagaaaatccgggtccaatgtctgaactcatcaaagaaaatatgcatatgaaactgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggaagcggagctactaacttcagcctgctgaagcaggctggagacgtggaggagaaccctggacctggatccactagtatggcgcacgctgggagaacggggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaactcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaatataaa

SEQ ID NO: 7 - DNA, 12197 bp, Artificial Sequence
  synthetic polynucleotide (pTRIP-SFFV-CD19trunc- 2A-tagBFP-2A-BCL2)
acataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcgggctagcgccaccatggctctgcccgttactgcactccttctcccactcgctcttctcctgcatgctgctcggcctgatattcaaatgactcaaaccactagtagcctttccgcttcactcggcgatcgggtgacaatttcctgtcgggcctcacaagatatctccaagtaccttaactggtaccaacaaaagcccgacggcaccgtaaagctgttgatatatcacaccagtcgcttgcatagcggcgtgccgagccggttttcaggatccggcagtgggactgactacagtttgacgatatccaatttggaacaggaagacatagctacgtatttctgtcagcaaggcaacactctgccttatacctttggcggcggaactaaactcgaaattactggaggaggcggttccggtggaggaggcagcggggggggtggctcagaagtcaagttgcaagaaagcggcccaggactcgttgccccgtcacaatccctctctgtgacttgcacagtgtctggcgtatctcttccagattacggggtgagttggataagacaaccacctcggaaaggactcgaatggctcggcgtcatctggggatccgagactacgtattacaactctgcactgaagagccggctcacaattattaaagataatagtaaatcccaggtgtttctgaagatgaatagcctgcagacggacgataccgcaatctattattgcgcaaagcactactattatggcgggtcatacgccatggattattggggtcagggtactagcgttacggtgagtagcacaactaccccggcacccaggccgcctacgccagccccaactattgcttctcaacctctttctcttagacctgaagcctgtagacctgcagccggcggtgcggtccatacccggggactcgattttgcttgcgacatttatatttgggctcctctggctggaacctgcggcgtgctgctgctcagtcttgtcattactctgtattgtggtagtggtgccacaaacttttcactcctcaaacaagccggtgatgttgaagaaaatccgggtccaatgtctgaactcatcaaagaaaatatgcatatgaaactgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggaagcggagctactaacttcagcctgctgaagcaggctggagacgtggaggagaaccctggacctggatccactagtatggcgcacgctgggagaacggggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaactcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaatataaa

SEQ ID NO: 8 - DNA, 12533 bp, Artificial Sequence
  synthetic polynucleotide (pTRIP-SFFV-CD19z-2A- tagBFP-2A-BCL2)
acataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcgggctagcgccaccatggctctgcccgttactgcactccttctcccactcgctcttctcctgcatgctgctcggcctgatattcaaatgactcaaaccactagtagcctttccgcttcactcggcgatcgggtgacaatttcctgtcgggcctcacaagatatctccaagtaccttaactggtaccaacaaaagcccgacggcaccgtaaagctgttgatatatcacaccagtcgcttgcatagcggcgtgccgagccggttttcaggatccggcagtgggactgactacagtttgacgatatccaatttggaacaggaagacatagctacgtatttctgtcagcaaggcaacactctgccttatacctttggcggcggaactaaactcgaaattactggaggaggcggttccggtggaggaggcagcggggggggtggctcagaagtcaagttgcaagaaagcggcccaggactcgttgccccgtcacaatccctctctgtgacttgcacagtgtctggcgtatctcttccagattacggggtgagttggataagacaaccacctcggaaaggactcgaatggctcggcgtcatctggggatccgagactacgtattacaactctgcactgaagagccggctcacaattattaaagataatagtaaatcccaggtgtttctgaagatgaatagcctgcagacggacgataccgcaatctattattgcgcaaagcactactattatggcgggtcatacgccatggattattggggtcagggtactagcgttacggtgagtagcacaactaccccggcacccaggccgcctacgccagccccaactattgcttctcaacctctttctcttagacctgaagcctgtagacctgcagccggcggtgcggtccatacccggggactcgattttgcttgcgacatttatatttgggctcctctggctggaacctgcggcgtgctgctgctcagtcttgtcattactctgtattgtcgcgttaaatttagtcggtcagctgatgcacctgcttataaacaagggcaaaatcaactctacaatgaactgaacctcggccgccgcgaagaatatgacgtactcgataaacgcaggggtcgagatcccgaaatgggcgggaaaccccgacggaagaatccccaagagggtctctataacgagcttcaaaaggacaaaatggccgaagcatatagcgaaataggaatgaagggagaaaggcgtcgcggaaaaggtcatgacggtctgtatcaaggcttgtctaccgctacaaaagatacgtatgatgctttgcatatgcaagctctcccaccaagaggtagtggtgccacaaacttttcactcctcaaacaagccggtgatgttgaagaaaatccgggtccaatgtctgaactcatcaaagaaaatatgcatatgaaactgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatggaagcggagctactaacttcagcctgctgaagcaggctggagacgtggaggagaaccctggacctggatccactagtatggcgcacgctgggagaacggggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaactcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaatataaa

SEQ ID NO: 9 - DNA, 8686 bp, artificial sequence
  synthetic polynucleotide (pCDH1-CAR-CD3)
gcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagattctagagctagcgatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga